SEQUENCE LISTING (15 sequences)

SEQ ID NO: 1 - DNA, 5349 bp, synthetic construct
  misc_feature 1..5349: note = Synthetic: pAAV.hIgK.hRdCVF1L Shuttle vector sequence for mammalian expression cassette of human RdCVF1L (-Met) and human IgK signal sequence (+Asp) into rAAV
  source 1..5349: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattcgagccaccatggacatgagggtccctgctcagctcctggggctcctgctgctctggctctcaggtgccagatgtgatgccagcctgttcagcggccggatcctgatcaggaacaacagcgaccaggacgagctggacaccgaggccgaagtgagcaggaggctggagaacagactggtgctgctgttctttggcgccggagcctgccctcagtgccaggccttcgtgcccatcctgaaggatttctttgtgaggctgaccgacgagttctacgtgctgagagccgcccagctggccctggtgtatgtgagccaggacagcaccgaggagcagcaggacctgttcctgaaggacatgcccaagaagtggctgttcctgcccttcgaggacgacctgagaagagacctgggcaggcagttcagcgtggagagactgcccgccgtggtggtgctgaagcctgatggcgacgtgctgaccagagatggcgccgacgagatccagagactgggcaccgcctgcttcgccaactggcaggaggccgccgaggtcctggacagaaacttccagctgcccgaggatctggaggatcaggagcccagatccctgaccgagtgcctgaggcggcacaagtacagagtggagaaggccgccagaggcggcagagaccctggcggcggaggaggagaggagggcggagccggcggactgttctgatgagctagcaccggttgtacaagtcaagcggccaaccctccctagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 2 - DNA, 2752 bp, synthetic construct
  misc_feature 1..2752: note = Synthetic: rAAV2.hIgK.hRdCVF1L vector genomic DNA
  source 1..2752: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattcgagccaccatggacatgagggtccctgctcagctcctggggctcctgctgctctggctctcaggtgccagatgtgatgccagcctgttcagcggccggatcctgatcaggaacaacagcgaccaggacgagctggacaccgaggccgaagtgagcaggaggctggagaacagactggtgctgctgttctttggcgccggagcctgccctcagtgccaggccttcgtgcccatcctgaaggatttctttgtgaggctgaccgacgagttctacgtgctgagagccgcccagctggccctggtgtatgtgagccaggacagcaccgaggagcagcaggacctgttcctgaaggacatgcccaagaagtggctgttcctgcccttcgaggacgacctgagaagagacctgggcaggcagttcagcgtggagagactgcccgccgtggtggtgctgaagcctgatggcgacgtgctgaccagagatggcgccgacgagatccagagactgggcaccgcctgcttcgccaactggcaggaggccgccgaggtcctggacagaaacttccagctgcccgaggatctggaggatcaggagcccagatccctgaccgagtgcctgaggcggcacaagtacagagtggagaaggccgccagaggcggcagagaccctggcggcggaggaggagaggagggcggagccggcggactgttctgatgagctagcaccggttgtacaagtcaagcggccaaccctccctagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 3 - protein, 234 aa, synthetic construct
  REGION 1..234: note = Synthetic: Amino acid Sequence of Human RdCVF1L (-Met) and Human IgK signal sequence (+Asp)
MDMRVPAQLLGLLLLWLSGARCDASLFSGRILIRNNSDQDELDTEAEVSRRLENRLVLLFFGAGACPQCQAFVPILKDFFVRLTDEFYVLRAAQLALVYVSQDSTEEQQDLFLKDMPKKWLFLPFEDDLRRDLGRQFSVERLPAVVVLKPDGDVLTRDGADEIQRLGTACFANWQEAAEVLDRNFQLPEDLEDQEPRSLTECLRRHKYRVEKAARGGRDPGGGGGEEGGAGGLF

SEQ ID NO: 4 - protein, 233 aa, synthetic construct
  REGION 1..233: note = Synthetic: Amino acid Sequence of Human RdCVF1L (-Met) and Human IgK signal sequence
MDMRVPAQLLGLLLLWLSGARCASLFSGRILIRNNSDQDELDTEAEVSRRLENRLVLLFFGAGACPQCQAFVPILKDFFVRLTDEFYVLRAAQLALVYVSQDSTEEQQDLFLKDMPKKWLFLPFEDDLRRDLGRQFSVERLPAVVVLKPDGDVLTRDGADEIQRLGTACFANWQEAAEVLDRNFQLPEDLEDQEPRSLTECLRRHKYRVEKAARGGRDPGGGGGEEGGAGGLF

SEQ ID NO: 5 - protein, 131 aa, synthetic construct
  REGION 1..131: note = Synthetic: Amino acid Sequence of Human RdCVF1S (-Met) and Human IgK Signal Sequence (+Asp)
MDMRVPAQLLGLLLLWLSGARCDASLFSGRILIRNNSDQDELDTEAEVSRRLENRLVLLFFGAGACPQCQAFVPILKDFFVRLTDEFYVLRAAQLALVYVSQDSTEEQQDLFLKDMPKKWLFLPFEDDLRR

SEQ ID NO: 6 - protein, 130 aa, synthetic construct
  REGION 1..130: note = Synthetic: Amino acid Sequence of Human RdCVF1S (-Met) and Human IgK Signal Sequence
MDMRVPAQLLGLLLLWLSGARCASLFSGRILIRNNSDQDELDTEAEVSRRLENRLVLLFFGAGACPQCQAFVPILKDFFVRLTDEFYVLRAAQLALVYVSQDSTEEQQDLFLKDMPKKWLFLPFEDDLRR

SEQ ID NO: 7 - protein, 23 aa, synthetic construct
  REGION 1..23: note = Synthetic: Amino acid Sequence of Human IgK Signal Sequence (+Asp)
MDMRVPAQLLGLLLLWLSGARCD

SEQ ID NO: 8 - protein, 22 aa, synthetic construct
  REGION 1..22: note = Synthetic: Amino acid Sequence of Human IgK Signal Sequence
MDMRVPAQLLGLLLLWLSGARC

SEQ ID NO: 9 - protein, 23 aa, synthetic construct
  REGION 1..23: note = Synthetic: Amino acid Sequence of Human IgK Signal Sequence I
METDTLLWVLLLWVPGSTGDDIV

SEQ ID NO: 10 - DNA, 2752 bp, synthetic construct
  misc_feature 1..2752: note = Synthetic: Nucleotide Sequence of Human RdCVF1L (-Met) and Human IgK Signal Sequence I
  source 1..2752: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattcgagccaccatggagacggatacgttgctgtgggtcctcttgctgtgggtcccgggctctacaggagatgacatagtcgccagcctgttcagcggccggatcctgatcaggaacaacagcgaccaggacgagctggacaccgaggccgaagtgagcaggaggctggagaacagactggtgctgctgttctttggcgccggagcctgccctcagtgccaggccttcgtgcccatcctgaaggatttctttgtgaggctgaccgacgagttctacgtgctgagagccgcccagctggccctggtgtatgtgagccaggacagcaccgaggagcagcaggacctgttcctgaaggacatgcccaagaagtggctgttcctgcccttcgaggacgacctgagaagagacctgggcaggcagttcagcgtggagagactgcccgccgtggtggtgctgaagcctgatggcgacgtgctgaccagagatggcgccgacgagatccagagactgggcaccgcctgcttcgccaactggcaggaggccgccgaggtcctggacagaaacttccagctgcccgaggatctggaggatcaggagcccagatccctgaccgagtgcctgaggcggcacaagtacagagtggagaaggccgccagaggcggcagagaccctggcggcggaggaggagaggagggcggagccggcggactgttctgatgagctagcaccggttgtacaagtcaagcggccaaccctccctagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 11 - protein, 234 aa, synthetic construct
  REGION 1..234: note = Synthetic: Amino acid Sequence of Human RdCVF1L (-Met) and Human IgK Signal Sequence I
METDTLLWVLLLWVPGSTGDDIVASLFSGRILIRNNSDQDELDTEAEVSRRLENRLVLLFFGAGACPQCQAFVPILKDFFVRLTDEFYVLRAAQLALVYVSQDSTEEQQDLFLKDMPKKWLFLPFEDDLRRDLGRQFSVERLPAVVVLKPDGDVLTRDGADEIQRLGTACFANWQEAAEVLDRNFQLPEDLEDQEPRSLTECLRRHKYRVEKAARGGRDPGGGGGEEGGAGGLF

SEQ ID NO: 12 - DNA, 471 bp, synthetic construct
  misc_feature 1..471: note = Synthetic: Nucleotide Sequence of RdCVF2L
  source 1..471: mol_type = other DNA
atggttgacattctgggcgagcggcacctggtgacctgtaagggcgcgacggtggaggccgaggcggcgctgcagaacaaggtggtggcactgtacttcgcggcggcccggtgcgcgccgagccgcgacttcacgccgctgctctgcgacttctatacggcgctggtggccgaggcgcggcggcccgcgcccttcgaagtggtcttcgtgtcagccgacggcagctcccaggagatgctggacttcatgcgcgagctgcatggcgcctggctggcgctgcccttccacgacccctaccggcatgagctgaggaagaggtacaacgtcacagccatccccaagcttgtgattgtgaaacaaaatggggaggtcatcaccaacaaagggcggaagcagatccgggaacgggggttggcctgcttccaggactgggtggaggcggccgatatcttccagaatttctccgtttga

SEQ ID NO: 13 - protein, 156 aa, synthetic construct
  REGION 1..156: note = Synthetic: Amino Acid Sequence of RdCVF2L
MVDILGERHLVTCKGATVEAEAALQNKVVALYFAAARCAPSRDFTPLLCDFYTALVAEARRPAPFEVVFVSADGSSQEMLDFMRELHGAWLALPFHDPYRHELRKRYNVTAIPKLVIVKQNGEVITNKGRKQIRERGLACFQDWVEAADIFQNFSV

SEQ ID NO: 14 - DNA, 537 bp, synthetic construct
  misc_feature 1..537: note = Synthetic: Codon Optimized Nucleotide Sequence of RdCVF2L (-Met) and human IgK Signal Sequence (+Asp)
  source 1..537: mol_type = other DNA
atggacatgagggtccctgctcagctcctggggctcctgctgctctggctctcaggtgccagatgtgatgttgatatactgggagaaaggcatcttgtaacatgcaaaggggctacagtggaagctgaggctgcacttcagaacaaagtggtggctctgtatttcgctgctgcccggtgcgctccctcacgggactttacccccttgctgtgtgacttttacaccgccctggttgcggaagctagaaggccagccccttttgaggtggttttcgtcagtgctgatggttcaagccaagagatgctggactttatgagagagctgcatggcgcttggctcgcgttgccctttcacgacccctatagacacgagttgagaaagcggtacaacgtcaccgcaataccgaagcttgttatcgtaaaacaaaacggtgaggttatcaccaataaaggccgcaaacaaattcgcgagagaggcctggcgtgttttcaggattgggtggaagccgcagacatctttcagaacttttcagtgtga

SEQ ID NO: 15 - protein, 178 aa, synthetic construct
  REGION 1..178: note = Synthetic: Amino Acid Sequence of RdCVF2L (-Met) and human IgK Signal Sequence (+Asp)
MDMRVPAQLLGLLLLWLSGARCDVDILGERHLVTCKGATVEAEAALQNKVVALYFAAARCAPSRDFTPLLCDFYTALVAEARRPAPFEVVFVSADGSSQEMLDFMRELHGAWLALPFHDPYRHELRKRYNVTAIPKLVIVKQNGEVITNKGRKQIRERGLACFQDWVEAADIFQNFSV

(19) United States
(12) Patent Application Publication
(10) Pub. No.: US 2024/0342313 A1
(43) Pub. Date: Oct. 17, 2024

(54) VECTOR ENCODING ROD-DERIVED CONE VIABILITY FACTOR AND HUMAN IGK SIGNAL SEQUENCE

(71) Applicant: Pharma Cinq, LLC, Rockville, MD (US)

(72) Inventors: Tianci LUO, Fulton, MD (US); Daniel LIPUT, Olney, MD (US); Fong Qi LIANG, Mount Airy, MD (US)

(21) Appl. No.: 18/622,513

(22) Filed: Mar. 29, 2024

Related U.S. Application Data
(60) Provisional application No. 63/455,867, filed on Mar. 30, 2023.

(51) Int. Cl. A61K 48/00; A61P 27/02; C07K 14/47; C12N 9/02; C12N 15/86

(52) U.S. Cl. CPC A61K 48/0058; A61K 48/0083; A61P 27/02; C07K 14/47; C12N 9/0051; C12N 15/86; C12Y 108/01008; C07K 2319/02; C07K 2319/30; C12N 2750/14122; C12N 2750/14143; C12N 2750/14145; C12N 2750/14171

(57) ABSTRACT

The present invention relates to nucleic acids coding for and capable of expressing a full-length rod-derived cone viability factor (RdCVF) and human IgK signal sequence and viral vectors containing these nucleic acids. The invention also relates to compositions and pharmaceutical preparations comprising these nucleic acids or vectors, methods of producing or secreting a full-length RdCVF and human IgK signal sequence, and methods of treatment.

### REFERENCE TO ELECTRONIC SEQUENCE LISTING

The application contains a Sequence Listing which has been submitted electronically in .XML format and is hereby incorporated by reference in its entirety. Said .XML copy, created on Mar. 28, 2024, is named “0288-0066WO1.xml” and is 33,602 bytes in size. The sequence listing contained in this .XML file is part of the specification and is hereby incorporated by reference herein in its entirety.

### BACKGROUND OF THE INVENTION

Rod-derived cone viability factor (RdCVF) is a thioredoxin-like protein specifically expressed by rod photoreceptor cells in the retina (Léveillard et al. (2004) Nature Genetics 36:755-759 and the supplemental information). Two different RdCVF genes are found in humans and they are designated RdCVF1 and RdCVF2. Both RdCVF genes encode two products via alternative splicing: a full-length protein and a C-terminal post-transcriptionally truncated protein, known as RdCVF-long and RdCVF-short, respectively.

RdCVF1-short is described as a secreted trophic factor for promoting cone survival, and RdCVF1-Long as a redox-active enzyme that interacts with intracellular proteins (Léveillard et al. (2010) Sci Transl Med. 2(26): 26ps16). For example, tau is described as a binding partner for RdCVF1-L and tau is exclusively intracellular (Fridlich et al. (2009) Molecular & Cellular Proteomics 8(6):1206-18).

Citation or discussion of a reference herein shall not be construed as an admission that such is prior art to the present invention.

### SUMMARY OF THE INVENTION

In embodiments, the disclosure provides a nucleic acid encoding a full-length rod-derived cone viability factor (“RdCVF-long” or “RdCVFL”) protein and a nucleotide sequence encoding a human immunoglobulin kappa chain (IgK) signal sequence, wherein the human IgK signal sequence includes an amino acid sequence with at least 95% sequence identity to SEQ ID NO: 7.

In embodiments, the disclosure provides a nucleic acid as disclosed herein, wherein the RdCVFL protein is an RdCVF1L protein or an RdCVF2L protein.

In embodiments, the disclosure provides a nucleic acid, wherein the RdCVFL protein is an RdCVF1L protein.

In embodiments, the disclosure provides a nucleic acid as disclosed herein, wherein the RdCVFL protein is a human RdCVFL protein.

In embodiments, the disclosure provides a nucleic acid as disclosed herein, wherein the nucleotide sequence encoding the RdCVFL protein includes a recoded nucleotide sequence.

In embodiments, the disclosure provides a nucleic acid as disclosed herein, wherein the recoded nucleotide sequence lacks an initiating methionine codon.

In embodiments, the disclosure provides a nucleic acid as disclosed herein, wherein the human IgK signal sequence is N-terminal to the RdCVFL protein.

In embodiments, the disclosure provides a nucleic acid as disclosed herein, wherein the nucleotide sequence encoding the human IgK signal sequence is operatively linked to the nucleotide sequence encoding the human RdCVFL.

In embodiments, the disclosure provides a nucleic acid as disclosed herein, wherein the RdCVFL protein and the human IgK signal sequence include an amino acid sequence with at least 95% sequence identity to SEQ ID NO: 3 or SEQ ID NO: 15.

In embodiments, the disclosure provides a nucleic acid as disclosed herein, wherein the RdCVFL protein and the human IgK signal sequence include an amino acid sequence with at least 95% sequence identity to SEQ ID NO: 3.

In embodiments, the disclosure provides a nucleic acid as disclosed herein, wherein the recoded nucleotide sequence has at least 40% of the codons recoded.

In embodiments, the disclosure provides a nucleic acid as disclosed herein, wherein the recoded nucleotide sequence has at least 15% of the nucleotides different as compared to a corresponding native nucleotide sequence.

In embodiments, the disclosure provides a nucleic acid as disclosed herein, wherein the recoded nucleotide sequence is less than 90% identical to a corresponding native nucleotide sequence.

In embodiments, the disclosure provides a nucleic acid as disclosed herein, wherein the nucleotide sequence or recoded nucleotide sequence has one or more characteristics selected from no procarya inhibitory motifs, no consensus splice donor sites, no cryptic splice donor sites and the GC content is between 60-65%.

In embodiments, the disclosure provides a nucleic acid as disclosed herein, wherein a promoter sequence is operatively linked to the nucleotide sequence encoding the human IgK signal sequence and human RdCVFL protein.

In embodiments, the disclosure provides a nucleic acid as disclosed herein, wherein the promoter is selected from a phage lambda (PL) promoter; an SV40 early promoter; a herpes simplex viral (HSV) promoter; a cytomegalovirus (CMV) promoter; a hybrid promoter with CMV enhancer and chicken beta-actin promoter; a tetracycline-controlled trans-activator-responsive promoter (tet) system; a long terminal repeat (LTR) promoter, such as a MoMLV LTR, BIV LTR or an HIV LTR; a U3 region promoter of Moloney murine sarcoma virus; a Granzyme A promoter; a regulatory sequence(s) of the metallothionein gene; a CD34 promoter; a CD8 promoter; a thymidine kinase (TK) promoter; a B19 parvovirus promoter; a PGK promoter; a glucocorticoid promoter; a heat shock protein (HSP) promoter; an immunoglobulin promoter; an MMTV promoter; a Rous sarcoma virus (RSV) promoter; a lac promoter; a CaMV 35S promoter; a nopaline synthetase promoter; an MND promoter; and an MNC promoter.

In embodiments, the disclosure provides a nucleic acid as disclosed herein, wherein the promoter is a CMV promoter.

In embodiments, the disclosure provides a nucleic acid as disclosed herein, wherein the CMV promoter includes nucleotides 150-812 of SEQ ID NO: 2.

In embodiments, the disclosure provides a nucleic acid as disclosed herein, wherein an intron sequence is operatively linked to the sequence encoding the RdCVFL protein.

In embodiments, the disclosure provides a nucleic acid as disclosed herein, wherein the intron sequence is a beta-globin intron sequence.

In embodiments, the disclosure provides a nucleic acid as disclosed herein, wherein the intron sequence includes nucleotides 820-1312 of SEQ ID NO: 2.

In embodiments, the disclosure provides a nucleic acid as disclosed herein, wherein the nucleic acid includes SEQ ID NO: 2, the nucleotide sequence of 150-2044 of SEQ ID NO: 2 or the nucleotide sequences of 150-812, 820-1312 and 1340-2044 of SEQ ID NO: 2.

In embodiments, the disclosure provides a vector including the nucleic acid as disclosed herein.

In embodiments, the disclosure provides a vector as disclosed herein, wherein the vector is a non-viral vector.

In embodiments, the disclosure provides a vector as disclosed herein, wherein the non-viral vector is selected from a lipid nanoparticle (LNP), highly branched poly(β-amino ester) (HPAE), single-chain cyclic polymer (SCKP), poly(amidoamine) (PAMAM) dendrimer, and polyethyleneimine (PEI).

In embodiments, the disclosure provides a vector as disclosed herein, wherein the vector is a viral vector.

In embodiments, the disclosure provides a vector as disclosed herein, wherein the viral vector is selected from a retroviral vector, lentiviral vector, adenoviral vector, adeno-associated virus (AAV) vector, Herpes viral vector, hepatitis viral vector, SV40 vector, EBV vector and Newcastle disease virus vector.

In embodiments, the disclosure provides a vector as disclosed herein, wherein the viral vector is an adeno-associated virus (AAV) vector.

In embodiments, the disclosure provides a viral vector as disclosed herein, wherein the AAV vector is selected from AAV1, AAV2, AAV3, AAV4, AAV5, AAV6, AAV7, AAV8, AAV9 and AAVrh74.

In embodiments, the disclosure provides a viral vector as disclosed herein, wherein the AAV vector is AAV2.

In embodiments, the disclosure provides a viral vector as disclosed herein, wherein the AAV vector is AAV8.

In embodiments, the disclosure provides a viral vector as disclosed herein, wherein the viral vector is not a bovine immunodeficiency viral vector.

In embodiments, the disclosure provides an isolated cell including the nucleic acid as disclosed herein, wherein the cell is capable of secreting the RdCVFL protein.

In embodiments, the disclosure provides a method for producing the RdCVFL protein including culturing the cell under conditions that allow for expression and secretion of the RdCVFL protein and isolating the RdCVFL protein from the cell culture.

In embodiments, the disclosure provides a method as disclosed herein, further including purifying the RdCVFL protein from the cell culture.

In embodiments, the disclosure provides a method of secreting RdCVFL protein from a cell including administering to the cell the nucleic acid or the vector as disclosed herein under conditions permitting expression and secretion of RdCVFL encoded by the nucleic acid or vector.

In embodiments, the disclosure provides an isolated cell or the method as disclosed herein, wherein the cell is a mammalian cell.

In embodiments, the disclosure provides an isolated cell or the method as disclosed herein, wherein the cell is a human cell.

In embodiments, the disclosure provides an isolated cell or the method as disclosed herein, wherein the mammalian cell is an ocular cell.

In embodiments, the disclosure provides an isolated cell or the method as disclosed herein, wherein the ocular cell is selected from a retinal pigment epithelial (RPE) cell, a rod cell, a cone cell, a bipolar cell, a horizontal cell, an amacrine cell, a ganglion cell, and an ARPE-19 cell.

In embodiments, the disclosure provides an isolated cell or the method as disclosed herein, wherein the cell is in vitro.

In embodiments, the disclosure provides an isolated cell or the method as disclosed herein, wherein the cell is in vivo.

In embodiments, the disclosure provides an isolated cell or the method as disclosed herein, wherein the cell is ex vivo.

In embodiments, the disclosure provides an isolated cell or the method as disclosed herein, wherein the mammalian cell is selected from a 293 cell, a CHO cell, a PerC6 cell, a Vero cell, a BHK cell, a HeLa cell, a COS cell, a MDCK cell, a 3T3 cell and a WI38.

In embodiments, the disclosure provides an isolated cell or the method as disclosed herein, wherein the cells are encapsulated.

In embodiments, the disclosure provides a pharmaceutical preparation including (i) a pharmaceutically acceptable carrier and (ii) the nucleic acid as disclosed herein, the vector as disclosed herein, or (iii) a combination thereof.

In embodiments, the disclosure provides a method of preserving ocular rod and cone cells in the eye of a mammal including administering to the eye of the mammal the nucleic acid as disclosed herein, the vector as disclosed herein, the pharmaceutical composition as disclosed herein, or a combination thereof, in an amount effective to preserve the ocular rod and cone cells.

In embodiments, the disclosure provides a method as disclosed herein, wherein the vector or the nucleic acid is administered by subretinal injection.

In embodiments, the disclosure provides a method as disclosed herein, wherein about 5×10⁸ to about 1×10¹¹ vector genome copy (GC) of an AAV vector is administered by subretinal injection.

In embodiments, the disclosure provides a method as disclosed herein, wherein the vector or the nucleic acid is administered by intravitreal injection, injection to the intraanterior chamber of the eye, subconjunctival injection or subtenon injection.

In embodiments, the disclosure provides a method as disclosed herein, wherein about 5×10⁸ to about 5×10¹² vector genome copy (GC) of an AAV vector is administered by intravitreal injection.

In embodiments, the disclosure provides a method as disclosed herein, wherein the mammal is a human.

In embodiments, the disclosure provides a method as disclosed herein, wherein the mammal suffers from an ocular disease selected from the group consisting of a retinal dystrophy, Stargardt's disease, retinitis pigmentosa, dry age-related macular degeneration (dry AMD), geography atrophy (advanced stage of dry AMD), wet age-related macular degeneration (wet AMD), glaucoma/ocular hypertension, diabetic retinopathy, Bardet-Biedel syndrome, Bassen-Kornzweig syndrome, Best disease, choroidema, gyrate atrophy, congenital amaurosis, refsun syndrome, Usher syndrome, thyroid related eye disease, Grave's disease, a disease associated with retinal pigmented epithelial cells, anterior segment disease, lens disease/cataracts, an eye cup disorder, or uveitis.

In embodiments, the disclosure provides a method as disclosed herein, wherein before the administration the preserved ocular rod and cone cell does not contain the nucleic acid as disclosed herein.

In embodiments, the disclosure provides a method as disclosed herein, including administering to the eye of the mammal the nucleic acid as disclosed herein or the vector as disclosed herein, wherein the nucleic acid or the vector is administered by subretinal injection and the rod and cone cells are preserved at a site at least 1 mm from the site of the subretinal injection.

In embodiments, the disclosure provides a method, wherein the rod cells are preserved at a site at least 2 mm from the site of the subretinal injection.

In embodiments, the disclosure provides a method of treating a disease including administering to a mammal the nucleic acid as disclosed herein, the vector as disclosed herein, the pharmaceutical preparation as disclosed herein or a combination thereof, wherein the disease is a Central Nervous System (CNS) Disease.

In embodiments, the disclosure provides a method of treating a disease as disclosed herein, wherein the CNS Disease is Alzheimer's disease, Huntington's disease, Parkinson's disease or an olfactory disease.

In embodiments, the disclosure provides a method of treating a disease as disclosed herein, wherein the administration includes intrathecal injection.

In embodiments, the disclosure provides a method of treating a disease as disclosed herein, wherein about 5×10⁸ to about 5×10¹⁴ vector genome copy (GC) of an AAV vector is administered by intrathecal injection.

In embodiments, the disclosure provides a method of treating a disease as disclosed herein, wherein the administration includes intravenous injection.

In embodiments, the disclosure provides a method of treating a disease as disclosed herein, wherein about 5×10⁸ to about 1×10¹⁵ vector genome copy (GC) of an AAV vector is administered by intravenous injection.

In embodiments, the disclosure provides a method of treating a disease as disclosed herein, wherein the mammal is a human.

This summary of the invention does not necessarily describe all features or necessary features of the invention. The invention may also reside in a sub-combination of the described features.

### BRIEF DESCRIPTION OF THE FIGURES

For the purpose of illustrating the invention, there are depicted in the drawings certain embodiments of the invention. However, the invention is not limited to the precise arrangements and instrumentalities of embodiments depicted in the drawings.

FIG. 1 shows an SDS-PAGE silver stain analysis of purified recombinant AAV2-hIGK-hRdCVF1L vector particles. The presence of AAV particles was confirmed by visualization of the VP1, VP2 and VP3 capsid proteins.

FIG. 2 shows Western blot analysis of RdCVF1L expression and secretion in rAAV-RdCVF1L vector transduced ARPE-19 cells. A protein doublet immunoreactive to anti-RdCVF1L antibodies was detected at approximately 30 kDa (near the theoretical molecular weight of hRdCVF1L) in cell lysate and media from ARPE-19 cell cultures transduced with rAAV2.hIgK.hRdCVF1L, but not the control vector rAAV2.EGFP. The higher molecular weight band of the RdCVF1L doublet represents a glycosylated form of RdCVF1L.

FIG. 3 shows Western blot analysis of RdCVF1L expression and secretion with two different signal sequences in rAAV-RdCVF1l vector transduced HEK293 cells. A protein doublet immunoreactive to anti-RdCVF1L antibodies was detected at approximately 30 kDa (near the theoretical molecular weight of hRdCVF1L) in cell lysate and media from HEK293 cultures transduced with rAAV2.mIgK.hRdCVF1L, rAAV2.hIgK(-D).hRdCVF1L, rAAV2.hIgK.hRdCVF1L but not the control vector rAAV2.EGFP. Expression and secretion of RdCVF1L mediated by rAAV2.mIgK.hRdCVF1L and rAAV2.hIgK.hRdCVF1L vectors were much more efficient than by rAAV2.hIgK(-D).hRdCVF1L vector. rAAV2.mIgK.hRdCVF1L was a AAV vector encoding human RdCVF1L with a mouse IgK signal sequence; rAAV2.hIgK(-D).hRdCVF1L was a AAV vector encoding human RdCVF1L with a human IgK signal sequence (SEQ ID NO: 8); rAAV2.hIgK.hRdCVF1L was a AAV vector encoding human RdCVF1L with a human IgK signal sequence (+Asp) (SEQ ID NO: 7).

FIG. 4 shows photoreceptor rescue by subretinal injection of AAV-RdCVF1L in rd10 mice. Mouse retinal section showed the rescue of photoreceptor cells in the AAV2-RdCVF1L treated eye (Left Panel) as compared to the untreated contralateral eye (Right Panel). The arrow points to the location of photoreceptor cells (outer nuclear layer, ONL). The treated one had five ONL layers (Left Panel) and the untreated contralateral eye had only one ONL layer (Right Panel).

FIG. 5 shows RdCVF1L reduced Aβ 1-42 induced toxicity on SH-SY5Y cells. LDH release was measured in medium and cell lysate. LDH ratio (medium/total (medium plus lysate)) was normalized to % of vehicle control. SH-SY5Y cells were incubated with vehicle, RdCVF1L (175, 350 ng/mL) in serum free medium w/o β-Amyloid 1-42 at 0, 2.5, 5 and 10 μM for 24 hours. LDH was measured in medium and cell lysis. LDH ratio (medium/total), normalized to % of Vehicle Two ways ANOVA, +++P<0.001 vs vehicle, no β-A; ***P<0.001 vs vehicle with β-A 10 μM.

FIG. 6 shows RdCVF1L reduced Aβ 1-42 induced toxicity on NHNP cells. LDH release was measured in medium and cell lysate. LDH ratio (medium/total (medium plus lysate)) was normalized to % of vehicle control. Differentiated NHNP cells (8 days) were incubated with the vehicle, RdCVF1L (100, 200 ng/mL) in the medium+1% FBS w/o β-A 1-42 (Invitrogen, cat. 03-112) at 0 and 10 μM for 2 days. LDH was measured in medium and cell lysis. LDH ratio (medium/total), normalized to 1% of Vehicle Two Way ANOVA, +++<0.001 vs vehicle, no β-A; ***P<0.001 vs vehicle with β-A 10 μM.

### DETAILED DESCRIPTION

As used herein the transitional term “comprising” is open-ended. A claim utilizing this term can contain elements in addition to those recited in such claim. Thus, for example, the claims can read on methods that also include other steps not specifically recited therein, as long as the recited elements or their equivalent are present.

The terms “identity” and “identical” when used in the context of comparing two sequences, such as nucleotide or amino acid sequences, refers to the percentage of the sequence that aligns between the two sequences. Percent identity can be determined by algorithms commonly employed by those skilled in this art. For example, percent identity can be determined using tools and programs available from the National Center for Biotechnology Information (NCBI) as available on their website. The percent identity of two nucleotide sequences can be determined, for example, using the NCBI/BLAST/blastn suite. Blastn can be used with the parameters set at: expect threshold=10; word size=28; max matches in a query range=0; match/mismatch scores=1,−2; gap costs=existence:5 extension:2.

It is contemplated that any method or composition described herein can be implemented with respect to any other method or composition described herein. The use of the word “a” or “an” when used in conjunction with the term “comprising” in the claims and/or the specification may mean “one,” but it is also consistent with the meaning of “one or more,” “at least one,” and “one or more than one.” The use of the term/phrase “and/or” when used with a list means one or more of the listed items may be utilized, e.g., it is not limited to one or all of the elements.

### INTRODUCTION

Individuals suffering from some retinal dystrophies were found to have lower levels of RdCVF protein in their eyes than did individuals without retinal dystrophies (see, e.g., PCT Publication WO02/081513). Different forms of RdCVF protein promote cone photoreceptor cell survival in vitro and in vivo. For example, intraocular injections of the short form of human RdCVF1 (RdCVFIS) protein not only rescued cone cells from degeneration but also preserved their function in animal models of inherited retinal degeneration (Yang et al. (2009) Mol Therapy 17:787-795).

Despite the promise of RdCVF protein for treatment of a variety of diseases, including ocular diseases, expression of significant levels of RdCVF at large scale and from gene therapy vectors has been challenging, e.g., see U.S. Patent Publication No. 20110034546, paragraph [0004]. Indeed, the hydrophobic nature of the protein has hampered its scalable production and purification using standard method. See, e.g., Sahel J A, Novel Treatments for Vision Disorders. Research EU Results Magazine. September 2014 (cordis.europa.eu/project/id/241683/reporting).

The present disclosure provides nucleic acids and vectors that encode an RdCVF protein with a signal sequence that allows the large-scale production of RdCVF for use in therapy, and compositions and methods of treatment utilizing the same. In embodiments of the disclosure, large scale production of various forms of RdCVF is accomplished by nucleic acids that encode an RdCVF protein and a surprisingly superior human Immunoglobulin kappa chain (IgK) signal sequence. In embodiments, the use of the human IgK sequence results in the expression of a polypeptide that has surprisingly superior expression and secretion, observed as a doublet, which represents a glycosylated form and a non-glycosylated form of RdCVF1L in Western Blot analysis. This is significant because glycosylation is a post-translational modification indicating that the protein will be secreted through the endoplasmic reticulum and Golgi secretory pathway. In embodiments, the human IgK signal sequence comprises an amino acid sequence with at least 95%, 96%, 97%, 98%, 99% or 100% sequence identity to SEQ ID NO: 7 or SEQ ID NO: 8. In embodiments, the human IgK signal sequence comprises an amino acid sequence with at least 95%, 96%, 97%, 98%, 99% or 100% sequence identity to SEQ ID NO: 7. In embodiments, the present disclosure provides a nucleic acid that encodes a human RdCVF protein and human IgK signal sequence which is a not a naturally occurring protein.

In embodiments, the nucleic acid of the invention encodes an RdCVF1 protein or an RDCVF2 protein and an IgK signal sequence comprising an amino acid sequence with at least 95% sequence identity to SEQ ID NO: 7.

In embodiments, the disclosure provides a nucleic acid encoding a protein comprising human RdCVF1L protein (amino acids 24-234 of SEQ ID NO: 3) and a human IgK signal sequence, wherein the human IgK signal sequence comprises an amino acid sequence with at least 95% sequence identity to SEQ ID NO: 7.

In embodiments, the disclosure provides a nucleic acid encoding a protein comprising RdCVF2L protein (SEQ ID NO: 13) and an IgK signal sequence comprising an amino acid sequence with at least 95% sequence identity to SEQ ID NO: 7.

In embodiments, the nucleic acid of the invention encodes a long version RdCVF1 protein and an IgK signal sequence comprising an amino acid sequence of SEQ ID NO: 7. In embodiments, the nucleic acid of the invention encodes a long version RdCVF2 protein and an IgK signal sequence comprising an amino acid sequence of SEQ ID NO: 7.

In embodiments, the disclosure provides a nucleic acid that encodes an RdCVF protein and a human IgK signal sequence comprising an amino acid sequence with at least 95%, 96%, 97%, 98%, 99%, or 100% sequence identity to SEQ ID NO: 3.

In embodiments, the disclosure provides a nucleic acid that encodes an RdCVF protein and a human IgK signal sequence comprising an amino acid sequence with at least 95%, 96%, 97%, 98%, 99%, or 100% sequence identity to SEQ ID NO: 15.

In embodiments, the disclosure provides a nucleic acid encoding a protein comprising RdCVF1L protein (amino acids 24-234 of SEQ ID NO: 3) and an IgK signal sequence comprising an amino acid sequence with at least 95% sequence identity to SEQ ID NO: 9.

PCT publications WO2002/081513, WO2008/148860, WO2009/146183 and WO WO2013/63383 describe various compositions and methods related to RdCVF. In some cases, RdCVF related compositions and methods described in PCT publications WO2002/081513, WO2008/148860, WO2009/146183 and WO WO2013/63383 can be utilized, for example, by replacing an RdCVF encoding nucleic acid, vector or protein with those of the present invention e.g., nucleic acids and vectors comprising an RdCVF coding sequence and human IgK signal sequence.

### RdCVF

RdCVF protein promotes cone photoreceptor cell survival in vitro and in vivo. For example, intraocular injections of the short form of human RdCVF1 (RdCVF1S) protein not only rescued cone cells from degeneration but also preserved their function in animal models of inherited retinal degeneration. (Yang et al. (Mol Therapy (2009) 17:787-795 and the supplemental material). RdCVF is expressed by several cell types including rod photoreceptor cells in the retina (Léveillard et al. (2004) Nature Genetics 36:755-759).

Two different RdCVF genes are found in humans and other mammals and they are designated RdCVF1 and RdCVF2. Both RdCVF genes encode two products via alternative splicing: a full-length protein and a C-terminal truncated protein, known as RdCVF-long (“RdCVFL”) and RdCVF-short, respectively. As used herein, “RdCVF1L” refers to the full length RdCVF1 protein (amino acids 24 to 234 of SEQ ID NO: 3) and “RdCVF2L” refers to the full length RdCVF2 protein (SEQ ID NO:13).

In some embodiments, the disclosure provides a recoded RdCVF coding sequence. A recoded RdCVF coding sequence can encode for any RdCVF protein including any of those disclosed herein. Sequences for various RdCVF proteins can be found in PCT Publication Nos. WO2002081513 and WO2010029130; Chalmel et al. (BMC Molecular Biology (2007) 8:74 pp 1-12 and the supplemental information); Léveillard et al. (Nature Genetics (2004) 36:755-759 and the supplemental information); Yang et al. (Mol Therapy (2009) 17:787-795 and the supplemental material) and GenBank Accession Nos. NP_612463, AAH14127, Q96CM4, EAW84608, CAD67528, Q5VZ03, NP_001155097, NP_660326, CAM24748, CAM14247, AAH22521 and CAD67531.

In some embodiments, an RdCVF protein is a fragment or an analog of an RdCVF protein that retains a cone cell and/or a rod cell survival activity or protective effect. Methods for measuring these activities or effects are known in the art. For example, Léveillard et al. (Nature Genetics (2004) 36:755-759 and the supplemental information) describes related mouse models and in vitro methods for detecting RdCVF activity. An RdCVF protein or an RdCVF coded for by a nucleic acid, can have an amino acid sequence other than a naturally-occurring amino acid sequence. For example, an RdCVF protein that is not naturally-occurring may contain amino acids in addition to those found in a naturally occurring RdCVF protein (e.g., at the amino or carboxy terminus) and/or may contain single or multiple amino acid substitutions (e.g., conservative or non-conservative amino acid substitutions) as compared to a naturally-occurring RdCVF amino acid sequence. A conservative amino acid substitution generally should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991). Conservative substitutions include, but are not limited to, those from the following groupings: Acidic Residues Asp (D) and Glu (E); Basic Residues Lys (K), Arg (R), and His (H); Hydrophilic Uncharged Residues Ser (S), Thr (T), Asn (N), and Gln (Q); Aliphatic Uncharged Residues Gly (G), Ala (A), Val (V), Leu (L), and Ile (I); Non-polar Uncharged Residues Cys (C), Met (M), and Pro (P); Aromatic Residues Phe (F), Tyr (Y), and Trp (W); Alcohol group-containing residues S and T; Aliphatic residues I, L, V and M; Cycloalkenyl-associated residues F, H, W and Y; Hydrophobic residues A, C, F, G, H, I, L, M, R, T, V, W and Y; Negatively charged residues D and E; Polar residues C, D, E, H, K, N, Q, R, S and T; Positively charged residues H, K and R; Small residues A, C, D, G, N, P, S, T and V; Very small residues A, G and S; Residues involved in turn formation A, C, D, E, G, H, K, N, Q, R, S, P and T; and Flexible residues Q, T, K, S, G, P, D, E and R. In some embodiments of the invention, a non-naturally occurring RdCVF protein has additional amino acids at the amino terminus, e.g., additional amino acids from a signal peptide. In some embodiments, an RdCVF protein of the invention is initially translated from a nucleotide coding sequence with a signal peptide and in some cases all or part of the amino acids of the signal peptide are retained on an expressed and/or secreted RdCVF protein of the invention.

### Recoded RdCVF Coding Sequences

In embodiments, the present disclosure provides a nucleic acid comprising a nucleotide sequence encoding an RdCVF protein and a human IgK signal sequence, wherein the human IgK signal sequence comprises an amino acid sequence with at least 95% sequence identity to SEQ ID NO: 7 or SEQ ID NO: 8, wherein the RdCVF coding sequence comprises a recoded nucleotide sequence. In embodiments, the RdCVF recoded nucleotide sequence lacks an initiating methionine codon.

The term “recoded” or “recoded nucleotide sequence” means that at least one native codon is changed to a different codon that encodes for the same amino acid as the native codon. In some embodiments, a recoded RdCVF coding region has at least 2.5%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least or at least 95% of the codons recoded. In some embodiments, about 20-50%, 35-45%, 38-42% or 39-41% or the codons are recoded. In some embodiments, a recoded codon is replaced with a codon that is more prevalently used in humans. In some embodiments, at least 5%, at least 10%, at least 15%, at least 20%, at least 25%, at least 30%, at least 35%, at least 40%, at least 45%, at least 50% or at least 55% of the codons have been replaced with a codon that is more prevalently used in humans.

In some embodiments, a recoded sequence has between about 70-90%, about 75-85%, about 80-85% or about 82-85% identity with the corresponding native coding sequence. In some embodiments, a recoded nucleotide sequence has at least 15% of the nucleotides different as compared to a corresponding native nucleotide sequence. In some embodiments, a recoded nucleotide sequence is less than 90% identical to a corresponding native nucleotide sequence.

Recoding can also be used to change the chemical make-up of a DNA and/or an RNA coding sequence such as the guanine/cytosine (GC) percentage. In some embodiments, recoding of an RdCVF coding region raises the GC content to at least 60%. In some embodiments, a recoded RdCVF coding region has a GC percentage between 60-64% or 60.4%-63.5%.

Recoding can be used to change the secondary structure of mRNA. Recoding can also be used to remove or add particular motifs or sites to a coding sequence or nucleic acid molecule, such as procarya inhibitory motifs, consensus splice donor sites, cryptic splice donor sites or a combination thereof. In some embodiments, a recoded RdCVF coding sequence has less procarya inhibitory motifs, consensus splice donor sites, cryptic splice donor sites or a combination thereof than the native sequence. In some embodiments, a recoded RdCVF coding sequence contains no procarya inhibitory motifs, no consensus splice donor sites and/or no cryptic splice donor sites.

Hoover et al. (Nucleic Acids Res. (2002) 30:e43, pp 1-7); Fath et al. (PLoS ONE (2011) 6:e17596 pp 1-14); Graf et al. (J Virol (2000) 74:10822-10826; Raab et al. Syst Synth Biol (2010) 4:215-225; and U.S. Patent Application 20070141557 describe recoding coding regions.

In some embodiments of the invention, a recoded RdCVF coding sequence does not contain the initial RdCVF ATG codon and/or RdCVF stop codon (e.g., TAG). For example, an RdCVF recoded coding sequence can be operatively linked 5′ or 3′ to another coding sequence resulting in a protein comprising a heterologous amino acid sequence, N-terminal and/or C-terminal to the RdCVF amino acid sequence, respectively. In some of these embodiments, the initial RdCVF ATG codon and/or RdCVF stop codon may be deleted or present in the RdCVF coding region. For example, see SEQ ID NO: 3 and SEQ ID NO: 6. If another coding sequence is fused in frame at the 3′ end of an RdCVF coding region, then the native RdCVF stop codon will not typically be present at the end of the RdCVF coding sequence.

The term “operatively linked” (or “operably linked”) as used herein means, with reference to a juxtaposition of two or more components (such as sequence elements), that the components are arranged such that both components function normally and allow the possibility that at least one of the components can mediate a function that is exerted upon at least one of the other components. For example, a nucleic acid that is operatively linked to a promoter is in functional combination therewith, e.g., under transcriptional initiation regulation of the promoter.

In some embodiments, a recoded RdCVF coding sequence is a recoded sequence that codes for amino acids 24-234 of SEQ ID NO: 3.

### Signal Peptides/Secretion Signals

The protein secretory pathway is utilized by more than a quarter of the human proteome. Signal peptide plays an essential role in guiding a newly synthesized protein to translocate from cytosol through the secretory pathway to a different location, e.g., cell membrane for a transmembrane protein or outside of the cells for a secreted protein. Signal peptides are highly diverse. In humans alone, more than 3000 proteins have been identified so far that contain different signal peptides. The highly diverse primary sequences of these signal peptides suggest that they play a role in regulating the physiological level of secretion of a particular protein from the cell (Kober, L. (2013) Biotechnol. Bioeng., 110:1164-1173; Cho, HJ. (2019) J. Microbiol. Biotechnol., 29:304-310; Liaci and Forster (2021) Int. J. Mol. Sci. 2021, 22, 11871. https://doi.org/10.3390/ijms222111871; Kangro, K. (2022) J. Thromb. Haemost., 20:2379-2385). The level of expression and secretion of a given protein is heavily dependent on the choice of signal peptide (Knappskog, S. (2007) Biotechnology, 128:705-715). Simply adjoining a heterologous signal peptide to a mature protein sequence does not guarantee secreted expression, and expression of a protein when placed directly adjacent to a non-native signal peptide could be completely abrogated (Guler-Cane, G (2016) PLOS ONE|DOI:10.1371/journal.pone.0155340 May 19, 2016). To add another layer of complexity, the efficiency of a signal peptide in guiding a protein secretion is also affected by the amino acid of the mature protein.

Endogenous human RdCVF does not have an identifiable signal peptide. A mouse IgK signal peptide was demonstrated to mediate efficient secretion of RdCVFL (U.S. Pat. No. 9,265,813). The mouse IgK signal sequence was used to reduce potential immunogenicity of a human signal sequence in in vivo studies in mice. Since there is no significant homology between mouse and human IgK signal peptide, the disclosure provides human IgK signal peptides that are capable of mediating expression and secretion of RdCVFL. Utilization of a signal peptide of human origin is expected to avoid immunogenicity associated with a mouse IgK signal peptide when the gene therapy vector is tested in human studies.

Signal sequences are translated in frame as a peptide attached, typically, to the amino-terminal end of a polypeptide of choice. A secretory signal sequence will cause the secretion of the polypeptide from the cell by interacting with the machinery of the host cell. As part of the secretory process, this secretory signal sequence will typically be cleaved off or at least partially cleaved off. The term “signal sequence” also refers to a nucleic acid sequence encoding the signal peptide.

The structure of a typical signal peptide can include three distinct regions: (i) an N-terminal region that contains a number of positively charged amino acids (e.g., lysines and arginines); (ii) a central hydrophobic core region (h-region); (iii) a hydrophilic cleavage region (c-region) that contains the sequence motif recognized by the signal peptidase. (e.g., see von Heijne, G. (1983) Eur. J. Biochem., 133:17-21; von Heijne, G. (1985) J. Mol. Biol., 184:99-105; von Heijne, G. (1997) Protein Engineering (10):1-6). These signal peptides can be used in accordance with the invention. In some embodiments, the signal peptide is from an immunoglobulin such as an IgK.

A signal sequence can be a mammalian, murine or human signal sequence. In some embodiments, a nucleic acid or vector of the invention comprises nucleotides 1340-1408 of SEQ ID NO: 2 or 1340-1405 of SEQ ID NO: 2. In some embodiments, a signal sequence codes for an amino acid sequence comprising amino acids 1-23 of SEQ ID NO: 3 or comprises amino acids 1-22 of SEQ ID NO: 4. A nucleotide sequence coding for a signal peptide can be a wild-type sequence or it can be a recoded sequence.

In some embodiments of the invention, a signal peptide sequence is operatively linked at the N-terminal or C-terminal of an RdCVF, e.g., RdCVF1L or RdCVF2L. In some embodiments, the signal peptide directs transit of the protein to secretory pathways, e.g., to the endoplasmic reticulum (ER). In some embodiments, a signal peptide facilitates protein transport from the cytoplasm to destinations outside the cell. Signal peptide sequences may be selected from naturally occurring signal peptide sequences, derivatives thereof, or a synthetic designed sequence. In some embodiments, non-limiting parameters for a designed signal peptide sequences include a sequence of 3-40 residues, comprising a 3- to 20-residue hydrophobic core flanked by several relatively hydrophilic residues.

### Nucleic Acids

The invention includes nucleic acids comprising a nucleotide sequence encoding an RdCVF and includes vectors comprising these nucleic acids.

To ensure local and/or long-term expression of a nucleic acid of interest, some embodiments of the invention contemplate transducing a cell with a nucleic acid or vector encoding an RdCVF. The instant invention is not to be construed as limited to any one particular nucleic delivery method, and any available nucleic acid delivery vehicle with either an in vivo or in vitro nucleic acid delivery strategy, or the use of manipulated cells (such as the technology of Neurotech, Lincoln, RI, e.g., see U.S. Pat. Nos. 6,231,879; 6,262,034; 6,264,941; 6,303,136; 6,322,804; 6,436,427; 6,878,544) as well as nucleic acids of the invention encoding an RdCVF per se (e.g., “naked DNA”), can be used in the practice of the invention. Various delivery vehicles, such as vectors, can be used with the invention. For example, viral vectors, amphitropic lipids, cationic polymers, such as polyethylenimine (PEI) and polylysine, dendrimers, such as combburst molecules and starburst molecules, nonionic lipids, anionic lipids, vesicles, liposomes and other synthetic nucleic acid means of delivery (e.g., see U.S. Pat. Nos. 6,958,325 and 7,098,030; Langer, Science 249:1527-1533 (1990); Treat et al., in “Liposomes” in “The Therapy of Infectious Disease and Cancer”; and Lopez-Berestein & Fidler (eds.), Liss, New York, pp. 317-327 and 353-365 (1989); Wang et al. J Nanobiotechnol 21, 272 (2023)) “naked” nucleic acids and so on can be used in the practice of the instant invention.

In some embodiments, a nucleic acid molecule is used in which the RdCVF coding sequences and any other desired sequences are flanked by regions that promote homologous recombination at a desired site in the genome, thus providing for intrachromosomal expression of the RdCVF nucleic acid (Koller et al., (1989) Proc. Natl. Acad. Sci. USA 86:8932-8935; Zijlstra et al. (1989) Nature 342:435-438). Delivery of a nucleic acid into a patient may be either direct, in which case the patient is directly exposed to the nucleic acid or nucleic acid-carrying vector, or indirect, in which case, cells are first transformed with the nucleic acid in vitro, then transplanted into the patient.

A vector is a means by which a nucleic acid of interest (e.g., a therapeutic nucleic acid that can encode a therapeutic protein) is introduced into a target cell of interest. Methods for obtaining or constructing a vector of interest include, but are not limited to, standard gene manipulation techniques, sequencing reactions, restriction enzymes digests, polymerase reactions, PCR, PCR SOEing, ligations, recombinase reactions (e.g., Invitrogen's GATEWAY® technology) other enzymes active on nucleic acids, bacteria and virus propagation materials and methods, chemicals and reagents, site directed mutagenesis protocols and so on, as known in the art, see, for example, the Maniatis et al. text, “Molecular Cloning.”

Nucleic acids of the invention will typically comprise a promoter sequence operatively linked to human IgK signal sequence and human RdCVF coding sequence. A promoter may be a tissue specific promoter, a cell specific promoter, an inducible promoter, a repressible promoter, a constitutive promoter, a synthetic promoter or a hybrid promoter, for example. Examples of promoters useful in the constructs of the invention include, but are not limited to, a phage lambda (PL) promoter; an SV40 early promoter; a herpes simplex viral (HSV) promoter; a cytomegalovirus (CMV) promoter, such as the human CMV immediate early promoter; a hybrid promoter with CMV enhancer and chicken beta-actin promoter; a tetracycline-controlled trans-activator-responsive promoter (tet) system; a long terminal repeat (LTR) promoter, such as a MoMLV LTR, BIV LTR or an HIV LTR; a U3 region promoter of Moloney murine sarcoma virus; a Granzyme A promoter; a regulatory sequence(s) of the metallothionein gene; a CD34 promoter; a CD8 promoter; a thymidine kinase (TK) promoter; a B19 parvovirus promoter; a PGK promoter; a glucocorticoid promoter; a heat shock protein (HSP) promoter, such as HSP65 and HSP70 promoters; an immunoglobulin promoter; an MMTV promoter; a Rous sarcoma virus (RSV) promoter; a lac promoter; a CaMV 35S promoter; and a nopaline synthetase promoter. In some embodiments, a promoter is an MND promoter (Robbins et al., 1997, J. Virol. 71:9466-9474), or an MNC promoter, which is a derivative of the MND promoter in which the LTR enhancers are combined with a minimal CMV promoter (Haberman et al., J. Virol. 74(18):8732-8739, 2000). In some embodiments, an RdCVF coding sequence is operatively linked to a promoter sequence comprising nucleotide sequence 150-812 of SEQ ID NO: 2.

In some embodiments, a vector or nucleic acid of the invention comprises an intron, operatively linked to a coding sequence for an RdCVF protein. An intron can be from an RdCVF gene or be a heterologous intron. Heterologous introns are known and non-limiting examples include a human β-globin gene intron and a beta-actin intron. In some embodiments, an intron sequence is a human β-globin gene intron sequence. In some embodiments, an intron sequence comprises nucleotides 820-1312 of SEQ ID NO: 2.

In some embodiments, a nucleic acid of the invention comprises a nucleotide sequence encoding a coding sequence for an RdCVF protein, wherein the RdCVF coding sequence comprises a recoded nucleotide sequence. A nucleic acid can encode for an RdCVF1 protein and/or an RdCVF2 protein. In embodiments, the RdCVF protein is an RdCVF1-long (RdCVF1L) or RdCVF2-long (RdCVF2L) protein. In some embodiments, the RdCVF protein is a human RdCVF1-long, or RdCVF2-long protein.

Typically a mammalian nucleotide coding region starts with the nucleotide sequence ATG (initiating methionine codon), such as found in a human RdCVF coding region. As discussed herein, some embodiments of the invention provide a recoded RdCVF coding region and in some further embodiments the coding region is fused, in-frame with a second coding region, e.g., a coding sequence for a signal sequence. In some of these cases, the ATG nucleotide sequence is not necessarily at the start of the RdCVF coding region, e.g., the RdCVF coding region starts by coding for the second amino acid of the particular RdCVF protein. However, the ATG nucleotide sequence can be at the start of the RdCVF coding region, even when the RdCVF coding region is operatively linked to another coding region 5′ to the RdCVF coding region.

In some embodiments, a nucleic acid of the invention comprises SEQ ID NOs: 2. In some embodiments, a nucleic acid of the invention comprises nucleotides 150-812, 820-1312 and 1340-2044 of SEQ ID NO: 2.

In some embodiments a nucleic acid of the invention comprises a coding region for an RdCVF, wherein the RdCVF coding sequence has been recoded.

In some embodiments of the invention, a nucleic acid of the invention is in a vector, such as a viral vector.

### Viral Vectors

The invention includes viral vectors comprising the nucleic acid of the invention, e.g., a vector comprising a nucleic acid comprising a nucleotide sequence encoding an RdCVF protein with a human IgK signal sequence, wherein the human IgK signal sequence comprises an amino acid sequence with at least 95% sequence identity to SEQ ID NO: 7 or SEQ ID NO: 8. Examples of viral vectors useful in the present invention are described in PCT Publication No. WO08/106644 and U.S. Patent Publication No. US20100120665. In some embodiments, the invention is not limited to a particular viral vector. Viral vectors include, but are not limited to, retroviral vectors, lentiviral vectors, adenoviral vectors (see, for example, U.S. Pat. No. 7,045,344), AAV vectors (e.g., see U.S. Pat. No. 7,105,345), Herpes viral vectors (e.g., see U.S. Pat. Nos. 5,830,727 and 6,040,172), hepatitis (e.g., hepatitis D) viral vectors (e.g., see U.S. Pat. No. 5,225,347), SV40 vectors, EBV vectors (e.g., see U.S. Pat. No. 6,521,449) and Newcastle disease virus vectors (e.g., see U.S. Pat. Nos. 6,146,642, 7,442,379, 7,332,169 and 6,719,979). In some embodiments, a lentiviral vector is an HIV, EIAV, SIV, FIV or BIV vector. In some embodiments, a vector is selected from an AAV vector or an adenoviral vector.

The invention also provides a cell that produces a viral vector of the invention. In embodiments, the cell that produces a viral vector of the invention is a 293 cell, a CHO cell, a PerC6 cell, a Vero cell, a BHK cell, a HeLa cell, a COS cell, a MDCK cell, a 3T3 cell and a WI38.

Vector virions of the invention may be administered in vivo or in vitro to cells (e.g., mammalian cells). Vectors (viral or nonviral) can be used to transduce or transform cells including, but not limited to, undifferentiated cells, differentiated cells, somatic cells, primitive cells and/or stem cells.

In some embodiments, a viral vector of the invention comprises a decay accelerating factor (DAF). For example, an enveloped viral vector includes a DAF on the viral membrane. In some embodiments, a DAF is a wild-type DAF. In some embodiments, a DAF is part of a fusion protein with an envelope protein, e.g., see Guibinga et al. Mol Ther. 2005 11(4):645-51.

Adenovirus is a non-enveloped, nuclear DNA virus with a genome typically of about 36 kb. The human adenoviruses are divided into numerous serotypes (approximately 47, numbered accordingly and classified into 6 groups: A, B, C, D, E and F).

Recombinant adenoviral vectors have tropism for both dividing and non-dividing cells, minimal pathogenic potential, ability to replicate to high titer for preparation of vector stocks and the potential to carry relatively large nucleotide sequence inserts (Berkner, (1992) Curr. Top. Micro. Immunol. 158:39-66; Jolly, (1994) Cancer Gene Therapy 1:51-64). Adenoviral vectors with deletions of various adenoviral gene sequences have been designed as suitable vehicles for delivery of nucleic acids to cells. In some embodiments, an adenoviral vector of the invention is a helper dependent or a “gutless” adenoviral vector. Adenoviral vectors can be used that are deleted in one or more of the following genes: E1a, E1b, E2a, E2b and E3. Methods for conducting adenovirus-based nucleic acid delivery are described in, e.g., U.S. Pat. Nos. 5,824,544; 5,868,040; 5,871,722; 5,880,102; 5,882,877; 5,885,808; 5,932,210; 5,981,225; 5,994,106; 5,994,132; 5,994,134; and 6,001,557.

AAV vectors are derived from single-stranded (ss) DNA parvoviruses. A single AAV particle can accommodate up to 5 kb of ssDNA, leaving about 4.5 kb for a transgene and regulatory elements. Trans-splicing systems as described, for example, in U.S. Pat. No. 6,544,785, may nearly double this limit and these types of vectors may also be used with the invention. With regard to the invention, essentially AAV of any serotype can be used. In some embodiments of the invention, an AAV1, AAV2, AAV3, AAV4, AAV5, AAV6, AAV7, AAV8, AAV9 or AAVrh74 serotype may be used (e.g., see U.S. Pat. Nos. 5,173,414, 5,252,479, 5,552,311, 5,658,776, 5,658,785, 5,763,416, 5,773,289, 5,843,742, 5,869,040, 5,942,496, 5,948,675, 6,001,650 and 7,790,449; PCT Publication No. WO2009134681; Kassim et al., PLoS ONE (2010) 5(10)e13424:1-10; Kotin, Hum Mol Genet (2011) 20 (R1):R2-6; Shoti et al., Mol Ther Methods Clin Dev (2023) 31:101147), although the invention is not limited to these serotypes (see, e.g., Gao et al. (2002) PNAS 99:11854-11859; and Viral Vectors for Gene Therapy: Methods and Protocols, ed. Machida, Humana Press, 2003).

An AAV vector of the invention may also be pseudotyped. Pseudotyped AAV vectors contain the genome of one AAV serotype in the capsid of a second AAV serotype (e.g., see Auricchio et al., (2001) Hum. Mol. Genet., 10(26):3075-81). An AAV vector of the invention may contain a mutated capsid and/or be retargeted. For example, see Grieger et al. (Adv Biochem Eng Biotechnol. (2005) 99:119-45); Gongalves et al. (Mol Ther. (2006) 13(5):976-86); and Warrington et al. (J Virol. (2004) 78(12):6595-609).

In some embodiments of the invention, an AAV vector is coated with polymers, e.g., reactive polymers to reduce natural tropism or natural binding of the AAV vector; to retarget the AAV vector and/or to provide resistance to neutralizing antisera. For example, see Carlisle et al. (J Gene Med. (2008) 10(4):400-11).

Further, AAV vectors derived from different serotypes of AAV can be modified in the capsid proteins by incorporating a peptide to enhance the vector's transduction efficiency, change the tropism for tissue and organ, reduce immunogenicity, and evade neutralizing antibodies against the viral vector. AAV capsid variable regions IV and VIII are permissive to capsid modification (Havlik et al., J. Virology, 94:e00976 (2020); Havlik et al., J. Virology, 95:e0058721 (2021); Becker et al., Pathogens, 11:756 (2022); Gonzalez et al., Nat Commun., 13:5947 (2022)).

Retroviruses are RNA viruses wherein the viral genome is RNA. When a host cell is infected with a retrovirus, the genomic RNA is reverse transcribed into a DNA intermediate which is integrated efficiently into the chromosomal DNA of infected cells. Lentiviruses contain other genes with regulatory or structural function. The use of retroviral vectors for gene delivery is described, for example, in U.S. Pat. Nos. 6,013,516; 5,994,136. Examples of BIV systems are described, for example, in Matukonis et al., 2002 Hum. Gene Ther. 13, 1293-1303; Molina et al., 2002 Virology. 304, 10-23; Molina et al., 2004 Hum. Gene Ther., 15, 65-877; U.S. Pat. Nos. 6,864,085, 7,125,712, 7,153,512; PCT Publication No. WO08/106644 and U.S. Patent Publication No. US20100120665.

A DNA viral vector is a viral vector based on or derived from a virus that has a DNA based genome. A non-enveloped virus viral vector is a viral vector based on or derived from a virus that lacks a lipid-bilayer membrane.

In some embodiments, a viral vector of the invention is an AAV vector. In some embodiments, a viral vector of the invention is not a bovine immunodeficiency viral vector or it is not a lentiviral vector. In some embodiments, a viral vector is selected from the group consisting of a DNA viral vector, a non-enveloped viral vector and an adenoviral vector.

Tremendous success of lipid nanoparticles (LNP) delivery technology in COVID-19 vaccine has proven LNP's utility as a non-viral vector delivery platform. LNP has also been used to deliver mRNA, siRNA, antisense oligonucleotides, microRNA and DNA in various preclinical studies (Hald Albertsen et al., Adv Drug Deliv Rev. 188:114416 (2022)). Nucleic acid sequence coding RdCVF could be delivered via LNP technology.

### Cellular Delivery of RdCVF, Including Encapsulated Cells

Another approach to gene therapy or protein delivery involves transferring a gene to cells in vitro or ex vivo and then administering the cells to a mammal or patient. Transferring a nucleic acid to cells can be by any method, such as, transfection, microinjection, electroporation, cell fusion, chromosome-mediated gene transfer, microcell-mediated gene transfer, spheroplast fusion, lipofection, microparticle bombardment, calcium phosphate mediated transfection, viral vector or bacteriophage transduction and so on. Optionally, a selectable marker also can be introduced into the cells. If a selectable marker is utilized, the cells can be then placed under selection, e.g., to enhance expression and/or to isolate/select those cells that express the transferred coding region (see, e.g., Loeffler & Behr, Meth. Enzymol. 217:599-618 (1993); Cohen et al., Meth. Enzymol. 217:618-644 (1993); and Cline, Pharmac. Ther. 29:69-92 (1985)). Those cells can then be delivered to a patient directly or after encapsulation.

In some embodiments, a nucleic acid is introduced into a cell prior to in vivo administration of the resulting recombinant cell. In some embodiments, a technique can provide for the stable transfer of the nucleic acid to the cell, so that the nucleic acid is expressible by the cell and in some cases heritable and expressible by its cell progeny. Recombinant cells can be delivered to a patient by various methods. In some embodiments, an RdCVF protein is expressed from a cell via a regulatable, inducible and/or repressible promoter.

In some embodiments, a cell used is autologous, allogeneic or xenogeneic with regard to a patient. In some embodiments, autologous cells are manipulated ex vivo to cause them to contain a nucleic acid of the invention which allows the cell to produce or secrete an RdCVF protein and the cells are introduced back to the patient.

In some embodiments, cells are administered locally (e.g., in a joint, intravitreal, intraretinal, intracranially etc.) or systemically (e.g., i.v.).

In some embodiments, recombinant blood cells (e.g., hematopoietic stem and/or progenitor cells) are administered intravenously. In some embodiments, eye cells and/or pluripotential cells can be injected directly into the eye.

A stem- and/or progenitor cell which can be isolated and maintained in vitro can potentially be used in accordance with some embodiments of the invention. Such stem cells include, but are not limited, to hematopoietic stem cells (HSC), stem cells of epithelial tissues such as the skin and the lining of the gut, embryonic heart muscle cells, liver stem cells (see, e.g., WO 94/08598), and neural stem cells (e.g., Stemple and Anderson (1992) Cell 71:973-985). In some embodiments, the administered cell is a stem cell comprising a nucleic acid of the invention and is capable of expressing and secreting an RdCVF.

Encapsulated cells can allow controlled and/or continuous delivery of a protein, such as RdCVF, in vivo. In some embodiments, cells comprising a nucleic acid of the invention and expressing and/or secreting an RdCVF are encapsulated. In some embodiments, cells are encapsulated within a semipermeable membrane that allows diffusion of RdCVF through the membrane. More information related to encapsulated cells and encapsulated cell implants is found in Sieving et al. (Proc Natl Acad Sci USA, (2006) 103(10):3896-901); U.S. Pat. Nos. 7,115,257 and 7,820,195; and PCT Publication No. WO2011044216. In some embodiments of the invention, encapsulated cells that express an RdCVF protein are delivered to an animal.

In some embodiments, encapsulated cells are implanted into a mammal, e.g., implanted in the eye, brain or olfactory region. In some embodiments, encapsulated cells are retinal pigment epithelial cells, e.g., ARPE-19 (available from ATCC, Manassas, VA). In some embodiments, encapsulated cells are used to deliver RdCVF to the eye, e.g., to the back of the eye.

In some embodiments, an encapsulated cell implant of the invention is comprised of cells that are encapsulated in a section of semi-permeable hollow fiber membrane and the cells have been genetically modified to produce an RdCVF. In some embodiments, an encapsulated cell implant has a suture loop at one end to anchor it to the sclera in the vitreo-retinal body inside the eye. In some embodiments, an encapsulated cell implant is 3, 4, 5, 6, 7, 8, 9 or 10 mm in length.

### RdCVF Protein Secretion and Production

Nucleic acids and viral vectors of the invention can be used to express, produce and/or secrete an RdCVF from a cell. This expression, production and/or secretion can occur in vitro, in vivo or ex vivo.

Some embodiments of the invention provide methods of secreting an RdCVF protein from a cell comprising administering to the cell a nucleic acid and/or a viral vector of the invention. In some embodiments, the cell can be a mammalian cell, a human cell, an ocular cell, a retinal pigment epithelial (RPE) cell, a rod cell or a cone cell.

Some embodiments of the invention utilize vertebrate or mammalian cells. Examples of useful mammalian host cell lines are a monkey kidney CVI cell line transformed by SV40 (e.g., COS-7, ATCC CRL 1651); human embryonic kidney line (e.g., 293 or 293T cells including either cell line subcloned for growth in suspension culture, Graham et al., J. Gen Virol. 36:59 (1977) such as 293 Freestyle (Invitrogen, Carlsbad, CA)) or 293FT; baby hamster kidney cells (e.g., BHK, ATCC CCL 10); Chinese hamster ovary cells (CHO cells); Chinese hamster ovary cells/-DHFR (e.g., CHO, Urlaub et al., Proc. Natl. Acad. Sci. USA 77:4216 (1980)); mouse sertoli cells (e.g., TM4, Mather, Biol. Reprod. 23:243-251 (1980)); monkey kidney cells (e.g., CVI ATCC CCL 70); African green monkey kidney cells (e.g., VERO-76, ATCC CRL-1587); human cervical carcinoma cells (e.g., HELA, ATCC CCL 2); canine kidney cells (e.g., MOCK, ATCC CCL 34); CF2TH cells; buffalo rat liver cells (e.g., BRL 3A, ATCC CRL 1442); human lung cells (e.g., W138, ATCC CCL 75); human liver cells (e.g., Hep G2, HB 8065); mouse mammary tumor cells (e.g., MMT 060562, ATCC CCL51); TRI cells (Mather et al., Annals N.Y. Acad. Sci. 383:44-68 (1983)); MRC 5 cells; ARPE-19 cells (ATCC) and FS4 cells.

In some embodiments, a cell is selected from the group consisting of a 293 cell, a CHO cell, a PerC6 cell, a Vero cell, a BHK cell, a HeLa cell, a COS cell, a MDCK cell, a 3T3 cell or a WI38.

Some embodiments of the invention provide an isolated cell comprising a nucleic acid of the invention. In some embodiments, the nucleic acid is integrated into the cellular genome/DNA.

The invention also includes methods for producing an RdCVF protein comprising culturing a cell under conditions that allow for expression and secretion of the RdCVF protein and isolating the RdCVF protein from the cell culture, wherein the cell comprises a nucleic acid of the invention that codes for and allows the expression of the RdCVF protein, e.g., secretion of an RdCVF protein. In some embodiments, the nucleic comprises a nucleotide sequence comprises a coding sequence for an RdCVF protein, wherein the RdCVF coding sequence comprises a recoded sequence. The RdCVF protein can be an RdCVF 1 or 2 protein or be the long or short form. In some embodiments, these methods further comprise purification of the RdCVF protein from the cell and/or culture supernatant.

The invention also includes an RdCVF protein expressed by a cell from a nucleic acid of the invention. The invention also provides secreted forms of RdCVF proteins of the invention and compositions comprising a secreted RdCVF protein of the invention.

In some embodiments, an RdCVF protein expressed from a cell is purified to at least 90%, at least 93%, at least 95%, at least 98%, at least 99.5% or at least 99.9% pure in relation to total protein.

### Compositions, Formulations and Preparations

Some embodiments of the invention provide compositions, formulations or preparations, e.g., pharmaceutical compositions, containing a nucleic acid of the invention, a vector of the invention, a RdCVF protein of the invention, or any combination thereof.

Formulations (e.g., for injection) are generally, but not necessarily, biocompatible solutions of the active ingredient, e.g., comprising Hank's solution, Ringer's solution or phosphate buffered saline. In some embodiments, a formulation or pharmaceutical composition comprises one or more of the following: citrate, NaCl, potassium chloride (KCl), calcium chloride dihydrate (CaCl₂·2H₂O), magnesium chloride hexahydrate (MgCl₂·6H₂O), sodium acetate trihydrate (CH₃CO₂Na·3H₂O), sodium citrate dihydrate (C₆H₅O₇Na₃·2H₂O), sucrose, sodium hydroxide and/or hydrochloric acid (to adjust pH) and water. The preceding list includes some molecules that are listed as particular hydrates, e.g., dihydrate, trihydrate, hexahydrate, etc. It is understood that various hydrates of these compounds can be used in the invention and the invention is not limited to these particular hydrate forms of the listed molecules. In some embodiments, a formulation or pharmaceutical composition comprises one or more ingredients selected from the group consisting of histidine, MgCl₂, trehalose, a polysorbate, polysorbate 20, NaCl, sucrose, arginine and proline. In some embodiments, a formulation comprises one or more of the following: histidine; α, α-trehalose dehydrate; MgCl₂; a polysorbate such as polysorbate 20; and NaCl. In some embodiments, a formulation or pharmaceutical composition comprises one or more of the following: phosphate buffered saline (PBS) and pluronic F-68. In some embodiments, pluronic F-68 concentration can be 0.0001%, 0.001%, 0.005%, 0.01% or 0.1%.

Examples of suitable formulations and formulatory methods for a desired mode of administration may be found in Remington's Pharmaceutical Sciences, latest edition, Mack Publishing Co., Easton, PA and in U.S. Pat. No. 7,208,577.

In some embodiments, a composition for use in vivo contains a “carrier” or a “pharmaceutically acceptable carrier”. The term “carrier” refers to a diluent, adjuvant, excipient, or vehicle with which a nucleic acid, vector or protein of the invention is administered. The term “carrier” includes, but is not limited to, either solid or liquid material, which may be inorganic or organic and of synthetic or natural origin, with which an active component(s) of the composition is mixed or formulated to facilitate administration to a subject. Any other materials customarily employed in formulating a pharmaceutical are suitable. In embodiments, pharmaceutical carriers differ from typical solutions and suspensions in that they are specifically prepared for use in vivo to exclude substances that may be harmful to the host to whom the composition is administered (e.g., removal of bacterial toxins).

Examples of suitable liquid carriers include water and aqueous solutions containing oxygenated organic compounds such as ethanol. Buffers and other materials normally present in pharmaceutical preparations, such as flavoring and suspending agents, can also be present. In general, a suitable oil(s), saline, aqueous dextrose (glucose), and related sugar solutions and glycols such as propylene glycol or polyethylene glycols are typically suitable carriers for parenteral solutions. In some embodiments, solutions for parenteral administration contain a water-soluble salt of the active ingredient, suitable stabilizing agents, and if desirable or necessary, buffer substances. Antioxidizing agents such as sodium bisulfite, sodium sulfite, or ascorbic acid, either alone or combined, can be used as stabilizing agents. Also used are citric acid and its salts and sodium EDTA. In addition, parenteral solutions can contain preservatives, such as benzalkonium chloride, methyl- or propyl-paraben and chlorobutanol.

In embodiments, carriers are carbohydrates, including but not limited to trehalose, mannitol, glutathione, xylitol, sucrose, lactose and sorbitol. In embodiments, the formulations of the disclosure include, natural or synthetic surfactants, for example, DPPC (1,2-Didecanoyl-sn-glycero-3-phosphocholine), DOPE (1,2-Dioleoyl-sn-glycero-3-phosphoethanolamine), DSPC (1,2-Distearoyl-sn-glycero-3-phosphocholinez 1,2-Distearoyl-sn-glycero-3-phosphocholine) and DOPC (1,2-Dioleoyl-sn-glycero-3-phosphocholine). In embodiments, the formulations of the invention include polyethylene glycol. In embodiments, the formulations of the invention include dextrans, such as cyclodextran. In embodiments, the formulations of the invention include cyclodextrin, tertiary amines and/or beta-cyclodextrin. In embodiments, the formulations of the invention include enhancers, such as bile salts. In embodiments, the formulations of the invention include cellulose and cellulose derivatives. In embodiments, the formulations of the invention include amino acids. In embodiments, the formulations of the invention include liposomes, microcapsules or microspheres, inclusion complexes, or other types of carriers is contemplated.

In embodiments, the formulations of the invention include wetting and/or emulsifying agents, and/or pH buffering agents. In embodiments, the formulations of the invention include a solubilizing agent and/or a local anesthetic such as lignocaine to ease pain at the site of the injection.

In some embodiments, a pharmaceutical preparation or composition of the invention comprises a (i) pharmaceutically acceptable carrier and (ii) a nucleic acid of the invention, a viral vector of the invention, an RdCVF protein of the invention or any combination thereof.

### Treatment, Administration and Delivery

It has been demonstrated that an RdCVF protein can promote cone photoreceptor cell survival in vitro and in vivo. For example, intraocular injections of the short form of human RdCVF1 (RdCVF1S) protein not only rescued cone cells from degeneration but also preserved their function in animal models of inherited retinal degeneration. (Yang et al. (Mol Therapy (2009) 17:787-795 and the supplemental material). Expression of endogenous RdCVF1 is mainly restricted to the retina (Léveillard et al. (2004) Nature Genetics 36:755-759).

In embodiments, the disclosure provides methods of preserving ocular rod and/or cone cells comprising administering to the eye of a mammal a nucleic acid of the invention, a viral vector of the invention, an RdCVF protein of the invention, a pharmaceutical composition of the invention or a combination thereof. In embodiments, the term “preserved” means maintaining the function of the ocular rod cells, for example, the function is maintained at 100%, 90%, 85%, 80%, 75%, 70%, 65%, 60%, 55%, 50%, 45%, 40%, 35%, 30%, 25%, 20%, 15%, 10% or 5% of function of healthy ocular rod cells. In some embodiments, a vector, e.g., viral vector and/or nucleic acid of the invention is administered by subretinal injection, intravitreal injection, injection to the intraanterior chamber of the eye, subconjunctival injection, subtenon injection or any combination thereof. In some embodiments, the mammal to be treated is a domesticated mammal, such as a cat, dog and horse. In embodiments, the mammal to be treated is a human.

In embodiments, the disclosure provides methods of preserving ocular rod and/or cone cells comprising administering to the eye of a mammal a nucleic acid of the invention, a viral vector of the invention, an RdCVF protein of the invention, a pharmaceutical composition of the invention or a combination thereof. In embodiments, the term “preserved” means maintaining the function of the ocular rod and/or cone cells, for example, the function is maintained at 100%, 90%, 85%, 80%, 75%, 70%, 65%, 60%, 55%, 50%, 45%, 40%, 35%, 30%, 25%, 20%, 15%, 10% or 5% of function of healthy ocular rod and/or cone cells. In some embodiments, a viral vector and/or nucleic acid of the invention is administered by subretinal injection, intravitreal injection, injection to the intraanterior chamber of the eye, subconjunctival injection, subtenon injection or any combination thereof. In some embodiments, the mammal to be treated is a domesticated mammal, such as a cat, dog and horse. In embodiments, the mammal to be treated is a human.

In some embodiments, the mammal to be treated suffers from an ocular disease, such as a retinal dystrophy, Stargardt's disease, retinitis pigmentosa, dry age-related macular degeneration (dry AMD), geographic atrophy (advanced stage of dry AMD), wet age-related macular degeneration (wet AMD), glaucoma/ocular hypertension, diabetic retinopathy, Bardet-Biedel syndrome, Bassen-Kornzweig syndrome, Best disease, choroidema, gyrate atrophy, congenital amaurosis, refsun syndrome, Usher syndrome, thyroid related eye disease, Grave's disease, a disease associated with retinal pigmented epithelial cells, anterior segment disease, lens disease/cataracts, an eye cup disorder, or uveitis. In some embodiments, the preserved ocular rod and/or cone cell does not contain a nucleic acid and/or viral vector of the invention. For example, the preserved ocular cell is not preserved through transduction of the preserved ocular cell itself.

The present disclosure further provides a method of preserving the function of ocular rod and cone cells comprising administering to the eye of a mammal a nucleic acid and/or viral vector of the invention, wherein the nucleic acid and/or the viral vector is administered by subretinal injection and the rod cells and/or cones cells are preserved at a site at least 1 mm, at least 2 mm, at least 3 mm, at least 5 mm, at least 7 mm, at least 10 mm, at least 15 mm, at least 20 mm, at least 25 mm, or at least 2 cm from the site of the subretinal injection. For example and not wishing to be bound by theory, the cells transduced with the nucleic acid or viral vector at the subretinal injection site expresses and/or secrete an RdCVF-long protein which can provide an ocular rod and/or cone preserving effect at a site distant to the transduced cell or injection site.

It is understood that when introduction or administration of a nucleic acid or vector encoding an RdCVF protein is disclosed, that the disclosure also provides introducing or administering the RdCVF protein itself. It is understood that when introduction of an RdCVF protein is disclosed, the invention also discloses introducing a nucleic acid or vector encoding an RdCVF protein.

In some embodiments, compositions of the invention are administered locally or systemically. Useful routes of administration are described herein and known in the art. Methods of introduction or administration include, but are not limited to, intradermal, intramuscular, intraperitoneal, intravenous, subcutaneous, intranasal, intratracheal, topical, inhaled, transdermal, rectal, parenteral routes, epidural, intracranial, into the brain, intraventricular, subdural, intraarticular, intrathecal, intracardiac, intracoronary, intravitreal, subretinal, intraanterior chamber, suprachoroidal of the eye, locally on the cornea, subconjunctival, subtenon injection, by applying eyedrops, oral routes, via balloon catheter, via stent or any combinations thereof. Systemic administration may be, but is not limited to, by intravenous or intra-arterial injection or by transmucosal, subcutaneous, transdermal and/or intraperitoneal delivery.

In some embodiments, e.g., comprising administration to the eye, an RdCVF1L or RdCVF2L encoding vector or nucleic acid of the invention is administered about once every week, month, 2 months, 3 months, 6 months, 9 months, year, 18 months, 2 years, 30 months, 3 years, 5 years, 10 years or as needed. In some embodiments, e.g., comprising administration to the eye, an RdCVF encoding vector or nucleic acid of the invention is administered from about every 1 to 4 weeks, about every 4 to 8 weeks, about every 1 to 4 months, about every 3 to 6 months, about every 4 to 8 months, about every 6 to 12 months, about every 9 to 15 months, about every 12 to 18 months, about every 15 to 21 months, about every 18 to 24 months, about every 1 to 2 years, about every 1.5 to 3 years, about every 2 to 4 years, about every 3 to 5 years, about every 5 to 7 years, about every 7 to 10 years or about every 10 to 20 years. It is expected that administration of a vector coding for an RdCVF protein would be less frequent than administration of the RdCVF protein itself. In some embodiments of the invention, a pharmaceutical preparation comprises a vector encoding an RdCVF protein of the invention and the pharmaceutical preparation is administered only once to the patient.

In some embodiments, an RdCVF1L or RdCVF2L encoding vector or nucleic acid of the invention is administered by intravitreal or subretinal injection to a human eye. In some embodiments, about 15 μg to about 5 mg; about 15 μg to about 500 μg; about 100 μg to about 900 μg; about 300 μg to about 700 μg; about 500 μg to about 1 mg; about 1 mg to about 5 mg; about 1 mg; or about 500 μg of an RdCVF protein is administered by intravitreal or subretinal injection to a human eye.

In some embodiments, an RdCVF1L or RdCVF2L encoding vector or nucleic acid of the invention is administered by subretinal injection or intravitreal injection. In some embodiments, about 5×10⁸ to about 1×10⁹; about 5×10⁸ to about 7.5×10⁸; about 7.5×10⁸ to about 1×10⁹; about 6×10⁸ to about 9×10⁸; about 7×10⁸ to about 8×10⁸; about 5×10⁸; about 6×10⁸; about 7×10⁸; about 8×10⁸; about 9×10⁸; or about 1×10⁹; or about 1×10¹⁰; or about 1×10¹¹; or about 1×10¹² vector genome copy (GC) number of an AAV vector is administered by subretinal injection. In some embodiments, about 5×10⁸ to about 1×10¹⁰; about 5×10⁸ to about 5×10⁹; about 5×10⁸ to about 2×10⁹; about 2×10⁹ to about 5×10⁹; about 5×10⁹ to about 1×10¹⁰; about 5×10⁸ to about 1×10⁹; about 1×10⁹ to about 3×10⁹; about 3×10⁹ to about 6×10⁹; about 6×10⁹ to about 1×10¹⁰; about 1×10⁹ to about 1×10¹⁰; about 1×10¹⁰ to about 1×10¹¹; or 1×10¹¹ to about 1×10¹²; or 1×10¹² to about 5×10¹² GC of an AAV vector is administered by intravitreal injection. In some embodiments, about 5×10⁸ to about 1×10¹⁰; about 5×10⁸ to about 5×10⁹; about 5×10⁸ to about 2×10⁹; about 2×10⁹ to about 5×10⁹; about 5×10⁹ to about 1×10¹⁰; about 5×10⁸ to about 1×10⁹; about 1×10⁹ to about 3×10⁹; about 3×10⁹ to about 6×10⁹; about 6×10⁹ to about 1×10¹⁰; about 1×10⁹ to about 1×10¹⁰; about 1×10¹⁰ to about 1×10¹¹; 1×10¹¹ to about 1×10¹²; 1×10¹² to about 1×10¹³; 1×10¹³ to about 1×10¹⁴; 1×10¹⁴ to about 5×10¹⁴ GC of an AAV vector is administered by intrathecal injection. In some embodiments, about 5×10⁸ to about 1×10¹⁰; about 5×10⁸ to about 5×10⁹; about 5×10⁸ to about 2×10⁹; about 2×10⁹ to about 5×10⁹; about 5×10⁹ to about 1×10¹⁰; about 5×10⁸ to about 1×10⁹; about 1×10⁹ to about 3×10⁹; about 3×10⁹ to about 6×10⁹; about 6×10⁹ to about 1×10¹⁰; about 1×10⁹ to about 1×10¹⁰; about 1×10¹⁰ to about 1×10¹¹; 1×10¹¹ to about 1×10¹²; 1×10¹² to about 1×10¹³; 1×10¹³ to about 1×10¹⁴; 1×10¹⁴ to about 1×10¹⁵ GC of an AAV vector comprising the RdCVF1L or RdCVF2L protein is administered by intravenous injection. It is understood that the amount of AAV vector is sometimes measured in transducing units or in GC number. GC numbers are typically between 25-300 times higher than when the same AAV vector sample is measured for transducing units.

The disclosure provides methods of treating a disease comprising administering to a mammal a nucleic acid of the invention, a viral vector of the invention, an RdCVF protein of the invention, a pharmaceutical composition of the invention or a combination thereof, wherein the disease is a Central Nervous System (CNS) disease. In some embodiments, the Central Nervous System (CNS) disease to be treated is Alzheimer's disease, Huntington's disease, Parkinson's disease and an olfactory disease. In some embodiments, the viral vector of this invention is an AAV vector.

Alzheimer's disease (AD) is the most common age-related neurodegenerative disorder and the major form of dementia, affecting about 50 million people globally. The patient number with AD is expected to triple by 2050. The disease occurs mainly in patients over 65 years of age, and is marked by a gradual decline in cognitive function. It is a progressive disease beginning with mild memory loss and possibly leading to loss of the ability to carry on a conversation and cope with daily life. Currently there is no treatment that can cure AD although immunotherapy may improve or slow the progression of symptoms.

Pathologically this disease is characterized by the accumulation of extracellular (β-amyloid (Aβ) peptide plaques and intracellular neurofibrillary tangles (NFTs). Aβ peptide of 40 or 42 amino acids is produced by proteolytic cleavage of the amyloid precursor protein (APP), while NFTs are composed of hyperphosphorylated and misfolded tau protein. These neuropathological hallmarks are accompanied by profound neuroinflammation marked by astrocytic and microglial activation. It is commonly believed that the neurotoxicity of Aβ and NFTs in brain is responsible for synaptic failure and neuronal degeneration. Mutations in APP or Tau cause the cognitive impairment and/or neuronal loss in mouse models of AD.

Neurotrophic factor therapy is an approach for neuroprotection in the AD brain, representing an alternative to amyloid-modifying drugs for preventing neuronal degeneration and stimulating neuronal function in Alzheimer's disease, with the ability to delay the disease progression. Nerve growth factor (NGF) and brain-derived neurotrophic factor (BDNF) administration showed an improvement in learning and memory, and a prevention of neuronal death in mouse models of AD. Gene therapy offers a unique approach to treating this chronic disease as its ability to maintain sustained expression of these factors in brain. Indeed, gene therapy for AD using AAV to deliver NGF or BDNF is currently in clinical trials.

Rod and cone photoreceptors are specialized neurons that function in the initial step of vision (Molday and Moritz, J Cell Sci., 128:4039 (2015)). RdCVF proteins have neuroprotective activity and are not only a factor for cone and/or rod survival, but could be general neuron survival factors. The fact that the RdCVF administration preserves rod and cone function, and the success of the in vitro studies using human neuroblastoma cell lines and human neural progenitor cells disclosed herein, are predictive of a neuroprotective effect of administration of RdCVF. Therefore, the disclosure herein provides a method of protecting brain neurons, which is beneficial for the treatment of CNS diseases, such as Alzheimer's disease.

Endogenous RdCVF1 expression is mainly restricted to the outer nuclear layer containing rod and cone photoreceptor cells, and endogenous RdCVF expression could not be detected in other organs/tissues such as kidney, testis, spleen, intestine, lung, and cerebellum (Leveillard, T. (2004) Nature Genetics, 36:755-759). Using a proteomics approach 90 proteins were found to interact with RdCVFL including the microtubule-binding protein tau (Fridlich et al. Mol Cell Proteomics (2009) 8(6):1206-1218). Fridlich et al. demonstrated that the level of phosphorylation of TAU is increased in the retina of the Nxnl1−/−(RdCVF1−/−) mice as it is hyperphosphorylated in the brain of patients suffering from Alzheimer disease, presumably in some cases through oxidative stress. Fridlich et al. also showed that RdCVFL inhibits TAU phosphorylation. Cronin et al. (Cell Death and Differentiation (2010) 17:1199-1210) found that Nxnl1−/−(RdCVF1−/−) retinas contained aggregated TAU protein, as found in the brain of patients suffering from Alzheimer's disease.

Mice lacking RdCVF2 have impaired vision and olfaction. Normal mice express RdCVF2 in the olfactory epithelium. Jaillard et al. (ARVO meeting (2009) program #/poster #491/D636) reported that olfactory neurons were found to survive to a higher rate when cultured in the presence of RdCVF2. Jaillard et al. also compared RdCVF2−/− to control mice, by performing olfactory discrimination learning tests. By 12 months of age, the RdCVF2−/− mice failed to respond correctly to the stimulus.

Therefore based on the above, an RdCVF encoding nucleic acid, viral vector or RdCVF protein of the invention can be used to treat or ameliorate Alzheimer's disease, Huntington's disease, Parkinson's disease and olfactory diseases.

All publications, patents, patent applications and all GenBank sequences mentioned in this specification are herein incorporated by reference in their entirety into the specification to the same extent as if each individual publication, patent or patent application was specifically and individually indicated to be incorporated herein by reference. Also incorporated by reference is any supplemental information that was published along with any of the aforementioned publications, patents and patent applications. For example, some journal articles are published with supplemental information that is typically available online.

Whereas, particular embodiments of the invention have been described herein for purposes of description, it will be appreciated by those skilled in the art that numerous variations of the details may be made without departing from the invention as described in the appended claims.

### SEQUENCES

pAAV.hIgK.hRdCVF1L Shuttle vector sequence for
mammalian expression cassette of human RdCVFIL
(−Met) and human IgK signal sequence (+Asp)
into rAAV
SEQ ID NO: 1
CCTGCAGGCAGCTGCGCGCTCGCTCGCTCACTGAGGCCGCCCGGGCAAAGCCCGGGCGTCGGG
CGACCTTTGGTCGCCCGGCCTCAGTGAGCGAGCGAGCGCGCAGAGAGGGAGTGGCCAACTCCA
TCACTAGGGGTTCCTGCGGCCGCACGCGTGGAGCTAGTTATTAATAGTAATCAATTACGGGGT
CATTAGTTCATAGCCCATATATGGAGTTCCGCGTTACATAACTTACGGTAAATGGCCCGCCTG
GCTGACCGCCCAACGACCCCCGCCCATTGACGTCAATAATGACGTATGTTCCCATAGTAACGT
CAATAGGGACTTTCCATTGACGTCAATGGGTGGAGTATTTACGGTAAACTGCCCACTTGGCAG
TACATCAAGTGTATCATATGCCAAGTACGCCCCCTATTGACGTCAATGACGGTAAATGGCCCG
CCTGGCATTATGCCCAGTACATGACCTTATGGGACTTTCCTACTTGGCAGTACATCTACGTAT
TAGTCATCGCTATTACCATGGTGATGCGGTTTTGGCAGTACATCAATGGGCGTGGATAGCGGT
TTGACTCACGGGGATTTCCAAGTCTCCACCCCATTGACGTCAATGGGAGTTTGTTTTGCACCA
AAATCAACGGGACTTTCCAAAATGTCGTAACAACTCCGCCCCATTGACGCAAATGGGCGGTAG
GCGTGTACGGTGGGAGGTCTATATAAGCAGAGCTCGTTTAGTGAACCGTCAGATCGCCTGGAG
ACGCCATCCACGCTGTTTTGACCTCCATAGAAGACACCGGGACCGATCCAGCCTCCGCGGATT
CGAATCCCGGCCGGGAACGGTGCATTGGAACGCGGATTCCCCGTGCCAAGAGTGACGTAAGTA
CCGCCTATAGAGTCTATAGGCCCACAAAAAATGCTTTCTTCTTTTAATATACTTTTTTGTTTA
TCTTATTTCTAATACTTTCCCTAATCTCTTTCTTTCAGGGCAATAATGATACAATGTATCATG
CCTCTTTGCACCATTCTAAAGAATAACAGTGATAATTTCTGGGTTAAGGCAATAGCAATATTT
CTGCATATAAATATTTCTGCATATAAATTGTAACTGATGTAAGAGGTTTCATATTGCTAATAG
CAGCTACAATCCAGCTACCATTCTGCTTTTATTTTATGGTTGGGATAAGGCTGGATTATTCTG
AGTCCAAGCTAGGCCCTTTTGCTAATCATGTTCATACCTCTTATCTTCCTCCCACAGCTCCTG
GGCAACGTGCTGGTCTGTGTGCTGGCCCATCACTTTGGCAAAGAATTGGGATTCGAACATCGA
TTGAATTCGAGCCACCATGGACATGAGGGTCCCTGCTCAGCTCCTGGGGCTCCTGCTGCTCTG
GCTCTCAGGTGCCAGATGTGATGCCAGCCTGTTCAGCGGCCGGATCCTGATCAGGAACAACAG
CGACCAGGACGAGCTGGACACCGAGGCCGAAGTGAGCAGGAGGCTGGAGAACAGACTGGTGCT
GCTGTTCTTTGGCGCCGGAGCCTGCCCTCAGTGCCAGGCCTTCGTGCCCATCCTGAAGGATTT
CTTTGTGAGGCTGACCGACGAGTTCTACGTGCTGAGAGCCGCCCAGCTGGCCCTGGTGTATGT
GAGCCAGGACAGCACCGAGGAGCAGCAGGACCTGTTCCTGAAGGACATGCCCAAGAAGTGGCT
GTTCCTGCCCTTCGAGGACGACCTGAGAAGAGACCTGGGCAGGCAGTTCAGCGTGGAGAGACT
GCCCGCCGTGGTGGTGCTGAAGCCTGATGGCGACGTGCTGACCAGAGATGGCGCCGACGAGAT
CCAGAGACTGGGCACCGCCTGCTTCGCCAACTGGCAGGAGGCCGCCGAGGTCCTGGACAGAAA
CTTCCAGCTGCCCGAGGATCTGGAGGATCAGGAGCCCAGATCCCTGACCGAGTGCCTGAGGCG
GCACAAGTACAGAGTGGAGAAGGCCGCCAGAGGCGGCAGAGACCCTGGCGGCGGAGGAGGAGA
GGAGGGCGGAGCCGGCGGACTGTTCTGATGAGCTAGCACCGGTTGTACAAGTCAAGCGGCCAA
CCCTCCCTAGATCTACGGGTGGCATCCCTGTGACCCCTCCCCAGTGCCTCTCCTGGCCCTGGA
AGTTGCCACTCCAGTGCCCACCAGCCTTGTCCTAATAAAATTAAGTTGCATCATTTTGTCTGA
CTAGGTGTCCTTCTATAATATTATGGGGTGGAGGGGGGTGGTATGGAGCAAGGGGCAAGTTGG
GAAGACAACCTGTAGGGCCTGCGGGGTCTATTGGGAACCAAGCTGGAGTGCAGTGGCACAATC
TTGGCTCACTGCAATCTCCGCCTCCTGGGTTCAAGCGATTCTCCTGCCTCAGCCTCCCGAGTT
GTTGGGATTCCAGGCATGCATGACCAGGCTCAGCTAATTTTTGTTTTTTTGGTAGAGACGGGG
TTTCACCATATTGGCCAGGCTGGTCTCCAACTCCTAATCTCAGGTGATCTACCCACCTTGGCC
TCCCAAATTGCTGGGATTACAGGCGTGAACCACTGCTCCCTTCCCTGTCCTTCTGATTTTGTA
GGTAACCACGTGCGGACCGAGCGGCCGCAGGAACCCCTAGTGATGGAGTTGGCCACTCCCTCT
CTGCGCGCTCGCTCGCTCACTGAGGCCGGGCGACCAAAGGTCGCCCGACGCCCGGGCTTTGCC
CGGGCGGCCTCAGTGAGCGAGCGAGCGCGCAGCTGCCTGCAGGGGCGCCTGATGCGGTATTTT
CTCCTTACGCATCTGTGCGGTATTTCACACCGCATACGTCAAAGCAACCATAGTACGCGCCCT
GTAGCGGCGCATTAAGCGCGGCGGGTGTGGTGGTTACGCGCAGCGTGACCGCTACACTTGCCA
GCGCCCTAGCGCCCGCTCCTTTCGCTTTCTTCCCTTCCTTTCTCGCCACGTTCGCCGGCTTTC
CCCGTCAAGCTCTAAATCGGGGGCTCCCTTTAGGGTTCCGATTTAGTGCTTTACGGCACCTCG
ACCCCAAAAAACTTGATTTGGGTGATGGTTCACGTAGTGGGCCATCGCCCTGATAGACGGTTT
TTCGCCCTTTGACGTTGGAGTCCACGTTCTTTAATAGTGGACTCTTGTTCCAAACTGGAACAA
CACTCAACCCTATCTCGGGCTATTCTTTTGATTTATAAGGGATTTTGCCGATTTCGGCCTATT
GGTTAAAAAATGAGCTGATTTAACAAAAATTTAACGCGAATTTTAACAAAATATTAACGTTTA
CAATTTTATGGTGCACTCTCAGTACAATCTGCTCTGATGCCGCATAGTTAAGCCAGCCCCGAC
ACCCGCCAACACCCGCTGACGCGCCCTGACGGGCTTGTCTGCTCCCGGCATCCGCTTACAGAC
AAGCTGTGACCGTCTCCGGGAGCTGCATGTGTCAGAGGTTTTCACCGTCATCACCGAAACGCG
CGAGACGAAAGGGCCTCGTGATACGCCTATTTTTATAGGTTAATGTCATGATAATAATGGTTT
CTTAGACGTCAGGTGGCACTTTTCGGGGAAATGTGCGCGGAACCCCTATTTGTTTATTTTTCT
AAATACATTCAAATATGTATCCGCTCATGAGACAATAACCCTGATAAATGCTTCAATAATATT
GAAAAAGGAAGAGTATGAGTATTCAACATTTCCGTGTCGCCCTTATTCCCTTTTTTGCGGCAT
TTTGCCTTCCTGTTTTTGCTCACCCAGAAACGCTGGTGAAAGTAAAAGATGCTGAAGATCAGT
TGGGTGCACGAGTGGGTTACATCGAACTGGATCTCAACAGCGGTAAGATCCTTGAGAGTTTTC
GCCCCGAAGAACGTTTTCCAATGATGAGCACTTTTAAAGTTCTGCTATGTGGCGCGGTATTAT
CCCGTATTGACGCCGGGCAAGAGCAACTCGGTCGCCGCATACACTATTCTCAGAATGACTTGG
TTGAGTACTCACCAGTCACAGAAAAGCATCTTACGGATGGCATGACAGTAAGAGAATTATGCA
GTGCTGCCATAACCATGAGTGATAACACTGCGGCCAACTTACTTCTGACAACGATCGGAGGAC
CGAAGGAGCTAACCGCTTTTTTGCACAACATGGGGGATCATGTAACTCGCCTTGATCGTTGGG
AACCGGAGCTGAATGAAGCCATACCAAACGACGAGCGTGACACCACGATGCCTGTAGCAATGG
CAACAACGTTGCGCAAACTATTAACTGGCGAACTACTTACTCTAGCTTCCCGGCAACAATTAA
TAGACTGGATGGAGGCGGATAAAGTTGCAGGACCACTTCTGCGCTCGGCCCTTCCGGCTGGCT
GGTTTATTGCTGATAAATCTGGAGCCGGTGAGCGTGGGTCTCGCGGTATCATTGCAGCACTGG
GGCCAGATGGTAAGCCCTCCCGTATCGTAGTTATCTACACGACGGGGAGTCAGGCAACTATGG
ATGAACGAAATAGACAGATCGCTGAGATAGGTGCCTCACTGATTAAGCATTGGTAACTGTCAG
ACCAAGTTTACTCATATATACTTTAGATTGATTTAAAACTTCATTTTTAATTTAAAAGGATCT
AGGTGAAGATCCTTTTTGATAATCTCATGACCAAAATCCCTTAACGTGAGTTTTCGTTCCACT
GAGCGTCAGACCCCGTAGAAAAGATCAAAGGATCTTCTTGAGATCCTTTTTTTCTGCGCGTAA
TCTGCTGCTTGCAAACAAAAAAACCACCGCTACCAGCGGTGGTTTGTTTGCCGGATCAAGAGC
TACCAACTCTTTTTCCGAAGGTAACTGGCTTCAGCAGAGCGCAGATACCAAATACTGTCCTTC
TAGTGTAGCCGTAGTTAGGCCACCACTTCAAGAACTCTGTAGCACCGCCTACATACCTCGCTC
TGCTAATCCTGTTACCAGTGGCTGCTGCCAGTGGCGATAAGTCGTGTCTTACCGGGTTGGACT
CAAGACGATAGTTACCGGATAAGGCGCAGCGGTCGGGCTGAACGGGGGGTTCGTGCACACAGC
CCAGCTTGGAGCGAACGACCTACACCGAACTGAGATACCTACAGCGTGAGCTATGAGAAAGCG
CCACGCTTCCCGAAGGGAGAAAGGCGGACAGGTATCCGGTAAGCGGCAGGGTCGGAACAGGAG
AGCGCACGAGGGAGCTTCCAGGGGGAAACGCCTGGTATCTTTATAGTCCTGTCGGGTTTCGCC
ACCTCTGACTTGAGCGTCGATTTTTGTGATGCTCGTCAGGGGGGCGGAGCCTATGGAAAAACG
CCAGCAACGCGGCCTTTTTACGGTTCCTGGCCTTTTGCTGGCCTTTTGCTCACATGT

rAAV2.hIgK.hRdCVFIL vector genomic DNA
sequence
SEQ ID NO: 2
CCTGCAGGCAGCTGCGCGCTCGCTCGCTCACTGAGGCCGCCCGGGCAAAG
CCCGGGCGTCGGGCGACCTTTGGTCGCCCGGCCTCAGTGAGCGAGCGAGC
GCGCAGAGAGGGAGTGGCCAACTCCATCACTAGGGGTTCCTGCGGCCGCA
CGCGTGGAGCTAGTTATTAATAGTAATCAATTACGGGGTCATTAGTTCAT
AGCCCATATATGGAGTTCCGCGTTACATAACTTACGGTAAATGGCCCGCC
TGGCTGACCGCCCAACGACCCCCGCCCATTGACGTCAATAATGACGTATG
TTCCCATAGTAACGTCAATAGGGACTTTCCATTGACGTCAATGGGTGGAG
TATTTACGGTAAACTGCCCACTTGGCAGTACATCAAGTGTATCATATGCC
AAGTACGCCCCCTATTGACGTCAATGACGGTAAATGGCCCGCCTGGCATT
ATGCCCAGTACATGACCTTATGGGACTTTCCTACTTGGCAGTACATCTAC
GTATTAGTCATCGCTATTACCATGGTGATGCGGTTTTGGCAGTACATCAA
TGGGCGTGGATAGCGGTTTGACTCACGGGGATTTCCAAGTCTCCACCCCA
TTGACGTCAATGGGAGTTTGTTTTGCACCAAAATCAACGGGACTTTCCAA
AATGTCGTAACAACTCCGCCCCATTGACGCAAATGGGCGGTAGGCGTGTA
CGGTGGGAGGTCTATATAAGCAGAGCTCGTTTAGTGAACCGTCAGATCGC
CTGGAGACGCCATCCACGCTGTTTTGACCTCCATAGAAGACACCGGGACC
GATCCAGCCTCCGCGGATTCGAATCCCGGCCGGGAACGGTGCATTGGAAC
GCGGATTCCCCGTGCCAAGAGTGACGTAAGTACCGCCTATAGAGTCTATA
GGCCCACAAAAAATGCTTTCTTCTTTTAATATACTTTTTTGTTTATCTTA
TTTCTAATACTTTCCCTAATCTCTTTCTTTCAGGGCAATAATGATACAAT
GTATCATGCCTCTTTGCACCATTCTAAAGAATAACAGTGATAATTTCTGG
GTTAAGGCAATAGCAATATTTCTGCATATAAATATTTCTGCATATAAATT
GTAACTGATGTAAGAGGTTTCATATTGCTAATAGCAGCTACAATCCAGCT
ACCATTCTGCTTTTATTTTATGGTTGGGATAAGGCTGGATTATTCTGAGT
CCAAGCTAGGCCCTTTTGCTAATCATGTTCATACCTCTTATCTTCCTCCC
ACAGCTCCTGGGCAACGTGCTGGTCTGTGTGCTGGCCCATCACTTTGGCA
AAGAATTGGGATTCGAACATCGATTGAATTCGAGCCACCATGGACATGAG
GGTCCCTGCTCAGCTCCTGGGGCTCCTGCTGCTCTGGCTCTCAGGTGCCA
GATGTGATGCCAGCCTGTTCAGCGGCCGGATCCTGATCAGGAACAACAGC
GACCAGGACGAGCTGGACACCGAGGCCGAAGTGAGCAGGAGGCTGGAGAA
CAGACTGGTGCTGCTGTTCTTTGGCGCCGGAGCCTGCCCTCAGTGCCAGG
CCTTCGTGCCCATCCTGAAGGATTTCTTTGTGAGGCTGACCGACGAGTTC
TACGTGCTGAGAGCCGCCCAGCTGGCCCTGGTGTATGTGAGCCAGGACAG
CACCGAGGAGCAGCAGGACCTGTTCCTGAAGGACATGCCCAAGAAGTGGC
TGTTCCTGCCCTTCGAGGACGACCTGAGAAGAGACCTGGGCAGGCAGTTC
AGCGTGGAGAGACTGCCCGCCGTGGTGGTGCTGAAGCCTGATGGCGACGT
GCTGACCAGAGATGGCGCCGACGAGATCCAGAGACTGGGCACCGCCTGCT
TCGCCAACTGGCAGGAGGCCGCCGAGGTCCTGGACAGAAACTTCCAGCTG
CCCGAGGATCTGGAGGATCAGGAGCCCAGATCCCTGACCGAGTGCCTGAG
GCGGCACAAGTACAGAGTGGAGAAGGCCGCCAGAGGCGGCAGAGACCCTG
GCGGCGGAGGAGGAGAGGAGGGCGGAGCCGGCGGACTGTTCTGATGAGCT
AGCACCGGTTGTACAAGTCAAGCGGCCAACCCTCCCTAGATCTACGGGTG
GCATCCCTGTGACCCCTCCCCAGTGCCTCTCCTGGCCCTGGAAGTTGCCA
CTCCAGTGCCCACCAGCCTTGTCCTAATAAAATTAAGTTGCATCATTTTG
TCTGACTAGGTGTCCTTCTATAATATTATGGGGTGGAGGGGGGTGGTATG
GAGCAAGGGGCAAGTTGGGAAGACAACCTGTAGGGCCTGCGGGGTCTATT
GGGAACCAAGCTGGAGTGCAGTGGCACAATCTTGGCTCACTGCAATCTCC
GCCTCCTGGGTTCAAGCGATTCTCCTGCCTCAGCCTCCCGAGTTGTTGGG
ATTCCAGGCATGCATGACCAGGCTCAGCTAATTTTTGTTTTTTTGGTAGA
GACGGGGTTTCACCATATTGGCCAGGCTGGTCTCCAACTCCTAATCTCAG
GTGATCTACCCACCTTGGCCTCCCAAATTGCTGGGATTACAGGCGTGAAC
CACTGCTCCCTTCCCTGTCCTTCTGATTTTGTAGGTAACCACGTGCGGAC
CGAGCGGCCGCAGGAACCCCTAGTGATGGAGTTGGCCACTCCCTCTCTGC
GCGCTCGCTCGCTCACTGAGGCCGGGCGACCAAAGGTCGCCCGACGCCCG
GGCTTTGCCCGGGCGGCCTCAGTGAGCGAGCGAGCGCGCAGCTGCCTGCA
GG

Amino acid Sequence of Human RdCVFIL (−Met)
and Human IgK signal sequence (+Asp)
SEQ ID NO: 3
MDMRVPAQLLGLLLLWLSGARCDASLFSGRILIRNNSDQDELDTEAEVSR
RLENRLVLLFFGAGACPQCQAFVPILKDFFVRLTDEFYVLRAAQLALVYV
SQDSTEEQQDLFLKDMPKKWLFLPFEDDLRRDLGRQFSVERLPAVVVLKP
DGDVLTRDGADEIQRLGTACFANWQEAAEVLDRNFQLPEDLEDQEPRSLT
ECLRRHKYRVEKAARGGRDPGGGGGEEGGAGGLF

Amino acid Sequence of Human RdCVFIL (−Met)
and Human IgK signal sequence
SEQ ID NO: 4
MDMRVPAQLLGLLLLWLSGARCASLFSGRILIRNNSDQDELDTEAEVSRR
LENRLVLLFFGAGACPQCQAFVPILKDFFVRLTDEFYVLRAAQLALVYVS
QDSTEEQQDLFLKDMPKKWLFLPFEDDLRRDLGRQFSVERLPAVVVLKPD
GDVLTRDGADEIQRLGTACFANWQEAAEVLDRNFQLPEDLEDQEPRSLTE
CLRRHKYRVEKAARGGRDPGGGGGEEGGAGGLF

Amino acid Sequence of Human RdCVF1S (−Met)
and Human IgK Signal Sequence (+Asp)
SEQ ID NO: 5
MDMRVPAQLLGLLLLWLSGARCDASLESGRILIRNNSDQDELDTEAEVSR
RLENRLVLLFFGAGACPQCQAFVPILKDFFVRLTDEFYVLRAAQLALVYV
SQDSTEEQQDLFLKDMPKKWLFLPFEDDLRR

Amino acid Sequence of Human RdCVF1S (−Met)
and Human IgK Signal Sequence
SEQ ID NO: 6
MDMRVPAQLLGLLLLWLSGARCASLFSGRILIRNNSDQDELDTEAEVSRR
LENRLVLLFFGAGACPQCQAFVPILKDFFVRLTDEFYVLRAAQLALVYVS
QDSTEEQQDLFLKDMPKKWLFLPFEDDLRR

| | Amino acid Sequence of Human IgK |
|---|---|
| | Signal Sequence (+Asp) |
| SEQ ID NO: 7 | |
| | MDMRVPAQLLGLLLLWLSGARCD |

| | Amino acid Sequence of Human IgK |
|---|---|
| | Signal Sequence |
| SEQ ID NO: 8 | |
| | MDMRVPAQLLGLLLLWLSGARC |

| | Amino acid Sequence of Human IgK |
|---|---|
| | Signal Sequence I |
| SEQ ID NO: 9 | |
| | METDTLLWVLLLWVPGSTGDDIV |

Nucleotide Sequence of Human RdCVFIL
(−Met) and Human IgK Signal
Sequence I
SEQ ID NO: 10
CCTGCAGGCAGCTGCGCGCTCGCTCGCTCACTGAGGCCGCCCGGGCAAAG
CCCGGGCGTCGGGCGACCTTTGGTCGCCCGGCCTCAGTGAGCGAGCGAGC
GCGCAGAGAGGGAGTGGCCAACTCCATCACTAGGGGTTCCTGCGGCCGCA
CGCGTGGAGCTAGTTATTAATAGTAATCAATTACGGGGTCATTAGTTCAT
AGCCCATATATGGAGTTCCGCGTTACATAACTTACGGTAAATGGCCCGCC
TGGCTGACCGCCCAACGACCCCCGCCCATTGACGTCAATAATGACGTATG
TTCCCATAGTAACGTCAATAGGGACTTTCCATTGACGTCAATGGGTGGAG
TATTTACGGTAAACTGCCCACTTGGCAGTACATCAAGTGTATCATATGCC
AAGTACGCCCCCTATTGACGTCAATGACGGTAAATGGCCCGCCTGGCATT
ATGCCCAGTACATGACCTTATGGGACTTTCCTACTTGGCAGTACATCTAC
GTATTAGTCATCGCTATTACCATGGTGATGCGGTTTTGGCAGTACATCAA
TGGGCGTGGATAGCGGTTTGACTCACGGGGATTTCCAAGTCTCCACCCCA
TTGACGTCAATGGGAGTTTGTTTTGCACCAAAATCAACGGGACTTTCCAA
AATGTCGTAACAACTCCGCCCCATTGACGCAAATGGGCGGTAGGCGTGTA
CGGTGGGAGGTCTATATAAGCAGAGCTCGTTTAGTGAACCGTCAGATCGC
CTGGAGACGCCATCCACGCTGTTTTGACCTCCATAGAAGACACCGGGACC
GATCCAGCCTCCGCGGATTCGAATCCCGGCCGGGAACGGTGCATTGGAAC
GCGGATTCCCCGTGCCAAGAGTGACGTAAGTACCGCCTATAGAGTCTATA
GGCCCACAAAAAATGCTTTCTTCTTTTAATATACTTTTTTGTTTATCTTA
TTTCTAATACTTTCCCTAATCTCTTTCTTTCAGGGCAATAATGATACAAT
GTATCATGCCTCTTTGCACCATTCTAAAGAATAACAGTGATAATTTCTGG
GTTAAGGCAATAGCAATATTTCTGCATATAAATATTTCTGCATATAAATT
GTAACTGATGTAAGAGGTTTCATATTGCTAATAGCAGCTACAATCCAGCT
ACCATTCTGCTTTTATTTTATGGTTGGGATAAGGCTGGATTATTCTGAGT
CCAAGCTAGGCCCTTTTGCTAATCATGTTCATACCTCTTATCTTCCTCCC
ACAGCTCCTGGGCAACGTGCTGGTCTGTGTGCTGGCCCATCACTTTGGCA
AAGAATTGGGATTCGAACATCGATTGAATTCGAGCCACCATGGAGACGGA
TACGTTGCTGTGGGTCCTCTTGCTGTGGGTCCCGGGCTCTACAGGAGATG
ACATAGTCGCCAGCCTGTTCAGCGGCCGGATCCTGATCAGGAACAACAGC
GACCAGGACGAGCTGGACACCGAGGCCGAAGTGAGCAGGAGGCTGGAGAA
CAGACTGGTGCTGCTGTTCTTTGGCGCCGGAGCCTGCCCTCAGTGCCAGG
CCTTCGTGCCCATCCTGAAGGATTTCTTTGTGAGGCTGACCGACGAGTTC
TACGTGCTGAGAGCCGCCCAGCTGGCCCTGGTGTATGTGAGCCAGGACAG
CACCGAGGAGCAGCAGGACCTGTTCCTGAAGGACATGCCCAAGAAGTGGC
TGTTCCTGCCCTTCGAGGACGACCTGAGAAGAGACCTGGGCAGGCAGTTC
AGCGTGGAGAGACTGCCCGCCGTGGTGGTGCTGAAGCCTGATGGCGACGT
GCTGACCAGAGATGGCGCCGACGAGATCCAGAGACTGGGCACCGCCTGCT
TCGCCAACTGGCAGGAGGCCGCCGAGGTCCTGGACAGAAACTTCCAGCTG
CCCGAGGATCTGGAGGATCAGGAGCCCAGATCCCTGACCGAGTGCCTGAG
GCGGCACAAGTACAGAGTGGAGAAGGCCGCCAGAGGCGGCAGAGACCCTG
GCGGCGGAGGAGGAGAGGAGGGCGGAGCCGGCGGACTGTTCTGATGAGCT
AGCACCGGTTGTACAAGTCAAGCGGCCAACCCTCCCTAGATCTACGGGTG
GCATCCCTGTGACCCCTCCCCAGTGCCTCTCCTGGCCCTGGAAGTTGCCA
CTCCAGTGCCCACCAGCCTTGTCCTAATAAAATTAAGTTGCATCATTTTG
TCTGACTAGGTGTCCTTCTATAATATTATGGGGTGGAGGGGGGTGGTATG
GAGCAAGGGGCAAGTTGGGAAGACAACCTGTAGGGCCTGCGGGGTCTATT
GGGAACCAAGCTGGAGTGCAGTGGCACAATCTTGGCTCACTGCAATCTCC
GCCTCCTGGGTTCAAGCGATTCTCCTGCCTCAGCCTCCCGAGTTGTTGGG
ATTCCAGGCATGCATGACCAGGCTCAGCTAATTTTTGTTTTTTTGGTAGA
GACGGGGTTTCACCATATTGGCCAGGCTGGTCTCCAACTCCTAATCTCAG
GTGATCTACCCACCTTGGCCTCCCAAATTGCTGGGATTACAGGCGTGAAC
CACTGCTCCCTTCCCTGTCCTTCTGATTTTGTAGGTAACCACGTGCGGAC
CGAGCGGCCGCAGGAACCCCTAGTGATGGAGTTGGCCACTCCCTCTCTGC
GCGCTCGCTCGCTCACTGAGGCCGGGCGACCAAAGGTCGCCCGACGCCCG
GGCTTTGCCCGGGCGGCCTCAGTGAGCGAGCGAGCGCGCAGCTGCCTGCA
GG

Amino acid Sequence of Human RdCVFIL (−Met)
and Human IgK Signal Sequence I
SEQ ID NO: 11
METDTLLWVLLLWVPGSTGDDIVASLFSGRILIRNNSDQDELDTEAEVSR
RLENRLVLLFFGAGACPQCQAFVPILKDFFVRLTDEFYVLRAAQLALVYV
SQDSTEEQQDLFLKDMPKKWLFLPFEDDLRRDLGRQFSVERLPAVVVLKP
DGDVLTRDGADEIQRLGTACFANWQEAAEVLDRNFQLPEDLEDQEPRSLT
ECLRRHKYRVEKAARGGRDPGGGGGEEGGAGGLF

Nucleotide Sequence of RdCVF2L
SEQ ID NO: 12
ATGGTTGACATTCTGGGCGAGCGGCACCTGGTGACCTGTAAGGGCGCGAC
GGTGGAGGCCGAGGCGGCGCTGCAGAACAAGGTGGTGGCACTGTACTTCG
CGGCGGCCCGGTGCGCGCCGAGCCGCGACTTCACGCCGCTGCTCTGCGAC
TTCTATACGGCGCTGGTGGCCGAGGCGCGGCGGCCCGCGCCCTTCGAAGT
GGTCTTCGTGTCAGCCGACGGCAGCTCCCAGGAGATGCTGGACTTCATGC
GCGAGCTGCATGGCGCCTGGCTGGCGCTGCCCTTCCACGACCCCTACCGG
CATGAGCTGAGGAAGAGGTACAACGTCACAGCCATCCCCAAGCTTGTGAT
TGTGAAACAAAATGGGGAGGTCATCACCAACAAAGGGCGGAAGCAGATCC
GGGAACGGGGGTTGGCCTGCTTCCAGGACTGGGTGGAGGCGGCCGATATC
TTCCAGAATTTCTCCGTTTGA

Amino Acid Sequence of RdCVF2L
SEQ ID NO: 13
MVDILGERHLVTCKGATVEAEAALQNKVVALYFAAARCAPSRDFTPLLCD
FYTALVAEARRPAPFEVVFVSADGSSQEMLDFMRELHGAWLALPFHDPYR
HELRKRYNVTAIPKLVIVKQNGEVITNKGRKQIRERGLACFQDWVEAADI
FQNFSV

Codon Optimized Nucleotide Sequence of
RdCVF2L (−Met) and human IgK Signal
Sequence (+Asp)
SEQ ID NO: 14
ATGGACATGAGGGTCCCTGCTCAGCTCCTGGGGCTCCTGCTGCTCTGGCT
CTCAGGTGCCAGATGTGATGTTGATATACTGGGAGAAAGGCATCTTGTAA
CATGCAAAGGGGCTACAGTGGAAGCTGAGGCTGCACTTCAGAACAAAGTG
GTGGCTCTGTATTTCGCTGCTGCCCGGTGCGCTCCCTCACGGGACTTTAC
CCCCTTGCTGTGTGACTTTTACACCGCCCTGGTTGCGGAAGCTAGAAGGC
CAGCCCCTTTTGAGGTGGTTTTCGTCAGTGCTGATGGTTCAAGCCAAGAG
ATGCTGGACTTTATGAGAGAGCTGCATGGCGCTTGGCTCGCGTTGCCCTT
TCACGACCCCTATAGACACGAGTTGAGAAAGCGGTACAACGTCACCGCAA
TACCGAAGCTTGTTATCGTAAAACAAAACGGTGAGGTTATCACCAATAAA
GGCCGCAAACAAATTCGCGAGAGAGGCCTGGCGTGTTTTCAGGATTGGGT
GGAAGCCGCAGACATCTTTCAGAACTTTTCAGTGTGA

Amino Acid Sequence of RdCVF2L (−Met)
and human IgK Signal Sequence (+Asp)
SEQ ID NO: 15
MDMRVPAQLLGLLLLWLSGARCDVDILGERHLVTCKGATVEAEAALQNKV
VALYFAAARCAPSRDFTPLLCDFYTALVAEARRPAPFEVVFVSADGSSQE
MLDFMRELHGAWLALPFHDP

### YRHELRKRYNVTAIPKLVIVKQNGEVITNKGRKQIRERGLACFQDWVEAADIFQNFS V

### EXAMPLES

The invention is now described with reference to the following examples. These examples are provided for the purpose of illustration only and the invention should in no way be construed as being limited to these examples but rather should be construed to encompass any and all variations which become evident as a result of the teachings provided herein.

### Example 1—Expression of Human Rod-Derived Cone Viability Factor Long Containing a Human Immunoglobulin Light Chain Signal Peptide Mediated by Recombinant Adeno-Associated Virus

### Plasmid Cloning

The nucleotide sequence of human L1 IgK light chain leader sequence (hIgK) plus aspartic acid (D or Asp) was added to the N-terminus of a codon optimized human (RdCVF1L) open reading frame by PCR amplification from in house plasmid pAAV-LRd268 for the production of AAV vector encoding a protein of SEQ ID NO: 3 (RdCVF1L and human IgK signal sequence of SEQ ID NO:7). The 737 bp hIgK.hRdCVF1L PCR product and pAAV-LRr268 were digested with EcoR1 and Nhe1 and the hIgK.hRdCVF1L fragment was ligated into the 4627 bp fragment of pAAV-LRr268, creating plasmid pAAV.hIgK.hRdCVF1L (SEQ ID NO 1). The sequence identity of pAAV.hIgK.hRdCVF1L was confirmed by Sanger sequencing.

Production and Purification of rAAV2.hIgK.hRdCVF1L

HEK293FT cells were cultured in DMEM with GlutaMAX and supplemented with non-essential amino acids, sodium pyruvate, and 10% FBS and antibiotics (100 μg/mL penicillin and 50 μg/mL streptomycin). At ˜72 hours before transfection, cells were counted and seeded in eighteen 15-cm dishes at 1.5×106 cells per dish. On the day of transfection, the growth media was removed and replaced with fresh media. The cells were then transfected with PEI (1 μg/μL) using a PEI:DNA mass ratio of 2. Each 15-cm dish was transfected with 50.75 μg (0.35 μg/cm2) plasmid DNA in a 1:1:1 molar ratio of pRC2c, pHelper and rAAV2.hIgK.hRdCVF1L vectors. To prepare PEI-DNA complexes, plasmids were diluted in DMEM (2 mL/15-cm dish, no supplements) followed by PEI. The solution was mixed, incubated for 20 min at room temperature and gently mixed again before applying to cells. After ˜72 hours, the cells were scraped and the cells and media were collected and centrifuged at 1000×g for 5 min at 4° C. After centrifugation, the media was decanted and virus present in media was harvested by PEG precipitation by adding 5 g PEG 8000 and 0.3 g NaCl to each 50 mL of media, and incubating the media at 4° C. overnight. The cell pellet was resuspended in lysis buffer (dPBS+0.01% pluronic F68 and 200 mM NaCl) and lysed by 1× freeze thaw followed by sonication. The lysate was clarified by centrifugation at 3000×g for 30 min at 4° C. and the supernatant was collected. After incubation the PEG precipitation was centrifuged at 3000×g for 30 min at 4° C. and the media was discarded. The PEG pellet was resuspended in lysis buffer, combined with the clarified cell lysate and incubated with Benzonase (50 U/mL) for 60 min at 37° C. The lysate was then subjected to ultracentrifugation through an IDX (Iodixanol) density gradient. IDX was diluted in dPBS and underlaid in a 38.5 mL Quick-seal tub in the following order: 8 mL of 15% IDX with 1M NaCl, 6 mL of 25% IDX, 8 mL of 40% IDX and 5 mL of 60% IDX. The clarified lysate was layered on top of the IDX gradient and centrifuged in a T70i rotor at 60,000 RPM (264,904×g) for 180 min at 10° C. After centrifugation, 3 mL of the 40% IDX layer was collected by puncturing the ultracentrifuge tubes with an 18-g needle just below the 40-60% IDX interface. The collected IDX containing AAV particles was buffer exchanged by repeated dilution with formulation buffer (dPBS with 0.001% pluronic F68) and concentration with Amicon Ultra-15 100K Centrifugal Filters. After final concentration, the AAV sample was sterilized by centrifugation through a 0.22 μM filter, aliquoted and stored at −80° C.

qPCR Analysis for Genome Copy Titer Determination

Purified rAAV2.hIgK.hRdCVF1L titer, defined at genome copies (GC)/mL, was determined by qPCR using a standard curve method. A standard curve for calculating genome copy titer was generated by linearizing a pAAV.EGFP control vector with HindIII. The concentration of the gel purified linearized vector was determined by spectrophotometry and serial diluted from 2×10⁸ to 2×10³ copies/μL. Prior to performing qPCR, the rAAV sample was digested with DNase I to degrade any contaminating pAAV. hIgK.hRdCVF1L transfer vector. A 10 μL rAAV sample was diluted into 79 μL of nuclease-free H₂O, 10 μL of 10× DNase reaction buffer and 1 μL of DNase I, and incubated at 37° C. for 20 min and 75° C. for 10 min. The DNase I digested sample was serial diluted in 10 mM TRIS-HCl (1:10 and 1:100) and stored on ice for qPCR. Three 5 μL replicates of each sample dilution and standard curve concentration was added to 15 μL of iQ SYBR Green Supermix containing 0.67 μM FWD and REV primers targeting the CMV promoter of the rAAV transgene expression cassette. qPCR was performed on a Bio-RAD iCycle thermocycler using the following protocol: 3 min at 98° C., (melt for 15 sec at 98° C., anneal/extend for 30 sec at 58° C.)×40 cycles. A melt curve from 55° C. to 98° C. was performed to verify the specificity of PCR amplification. Following PCR, the amplification curves were baseline subtracted (automatically by iQ software) and the CT values, defined as the cycle number to reach threshold, were determined for each standard and unknown sample. A standard curve was plotted using the plasmid standards, fit by linear regression and the concentration of rAAV2.hIgK.hRdCVF1L dilution was determined. Titers were calculated according to the following equation: Titer (GC/mL)=quantity (calculated for standard curve)×10 (DNase I dilution)×2 (double stranded DNA standard)×dilution factor×1000 (convert μL to mL). The titer of rAAV2.hIgK.hRdCVF1L was 9.94×10¹² GC/mL.

Silver Stain Analysis for rAAV2.hIgK.hRdCVF1L

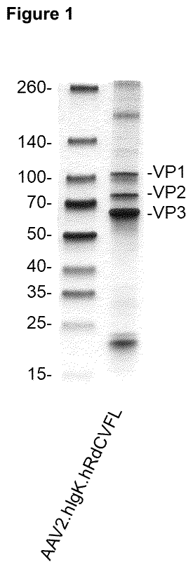

An aliquot of rAAV2.hIgK.hRdCVF1L containing 5×10⁹ genome copies was denatured in reducing sample buffer for 5 min at 95° C. and electrophoresed on a 4-15% polyacrylamide gel. The gel was then stained using the Pierce silver staining kit according to the manufacturer's instructions. Briefly, the gel was washed 2×5 min in ultrapure H₂O and fixed 2×15 min in 30% ethanol with 10% acetic acid. After fixation, the gel was washed 2×5 min in 10% ethanol and then 2×5 min in ultrapure H₂O. The gel was then incubated in sensitizer solution (50 μL sensitizer in 25 mL ultrapure H₂O) for 1 min and wash 2×1 min in ultrapure H₂O. Next, the gel was stained (0.5 mL enhancer in 25 mL stain) for 30 min and washed 2×20 sec in ultrapure H₂O. To develop, the gel was bathed in developer working solution (0.5 mL enhancer in 25 mL developer) until protein bands were evident. Development of the gel was stopped with 5% acetic acid for 10 min. The gel was washed in ultrapure H₂O and imaged using an Epson V700 PHOTO scanner. The presence of AAV particles was confirmed by visualization of the VP1, VP2 and VP3 capsid proteins (FIG. 1).

In Vitro Assay for rAAV2.hIgK.hRdCVF1L Gene Transfer

Protein expression and hRdCVF1L secretion following rAAV2.hIgK.hRdCVF1L transduction was evaluated in the human retinal pigmented epithelium cell line, APRE-19. ARPE-19 cells were plated in 1 mL of complete DMEM at 2×10⁵ cells per well, in a 6 well plate and incubated overnight. Cells were then transduced with either rAAV2.hIgK.hRdCVF1L or a rAAV2.EGFP control vector at an MOI of 1,000. After a 48-hour incubation the media was collected, centrifuged at 16,000×g for 30 min at 4° C. and stored at −20° C. The cells were harvested with dPBS−/− supplemented with 10 mM EDTA. The cells were centrifuged at 500×g for 5 min and the cell pellets were lysed with 75 μL ice cold RIPA buffer supplemented with Halt protease inhibitor cocktail. The cell lysates were incubated on ice for 30 min, centrifuged at 16,000×g for 30 min at 4° C. and stored at −20° C.

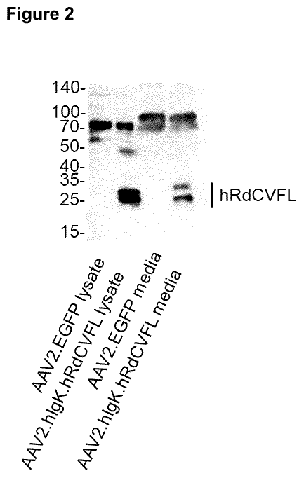

For western blotting, media samples and cell lysates were thawed on ice. Five microliters of each cell lysate was added to 11 μL dPBS with 4 μL 5× reducing sample buffer and denatured for 5 min at 95° C. For media samples, 16 μL was added to 4 μL 5× reducing buffer and denatured for 5 min at 95° C. Samples were electrophoresed on a 4-15% polyacrylamide gel and blotted to a nitrocellulose membrane. The nitrocellulose membrane was blocked in 1× casein for 1 hour at room temperature and incubated with primary rabbit anti-RdCVF1L (AD-10, 1:1000) antibodies in 1× casein over night at 4° C. After primary incubation, the membrane was washed 3×5 min in 1× casein, incubated in secondary biotinylated goat anti-rabbit antibodies (1:5,000) in 1× casein for 1 hour at room temperature. Following secondary incubation, the membrane was washed 3×5 min in 1× casein and incubated in Vectastain ABC-AMP (30 μL A and 30 μL B in 15 min 1× casein) for 40 min at room temperature. Next, the membrane was washed 3×5 min in 0.1M Tris-HCl (pH 9.5), incubated in Duolax for 5 min and washed again in 0.1 M Tris-HCl for 5 min. The membrane was exposed on BioMax light film for 180 sec and the film was subsequently bathed in developer solution (26 mL developer and replenisher solution in 92 mL H₂O) for 1 min and fixer solution (26 mL fixer and replenisher solution in 92 mL H₂O) for 1 min. The film was then rinsed in H₂O, dried and imaged using an Epson V700 PHOTO scanner. A protein doublet immunoreactive to anti-RdCVF1L antibodies was detected at approximately 30 KDa (near the theoretical molecular weight of hRdCVF1L) in cell lysate and media from ARPE-19 cell cultures transduced with rAAV2.hIgK.hRdCVF1L, but not the control vector rAAV2.EGFP (FIG. 2).

### Example 2—Comparison of rAAV Vector-Mediated Human RdCVF1L Expression and Secretion with a Different Human IgK Signal Sequence

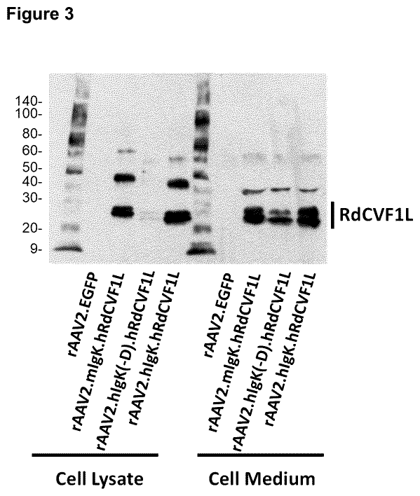

Recombinant AAV vector encoding human IgK signal sequence (+Asp) (SEQ ID NO: 7) and human RdCVF1L was described above in Example 1. A recombinant AAV vector encoding a protein of SEQ ID NO: 4 comprising human IgK signal sequence (−Asp) (SEQ ID NO: 8) and the same human RdCVF1L was generated, designated rAAV2.hIgK(-D).hRdCVF1L. The only difference between the two vectors, rAAV2.hIgK.hRdCVF1L and rAAV2.hIgK(-D).hRdCVF1L was one extra amino acid Aspartic Acid in the signal sequence in rAAV2.hIgK.hRdCVF1L vector. The two vectors were produced, tittered in identical way. Same amounts of the two vectors were used to transduce the same numbers of human HEK293FT cells. Seventy-two hours after transduction, the cell lysate and cell culture medium were processed as described above in Example 1 for expression and secretion of RdCVF1L by Western blot analysis. As shown in FIG. 3, the expression and secretion of human RdCVF1L were much more efficient by rAAV2.hIgK.hRdCVFIL vector as compared to rAAV2.hIgK(-D).hRdCVF1L vector. The data suggest that surprisingly even one amino acid difference in the signal sequence could have significant impact in protein expression and secretion (FIG. 3).

### Example 3—RdCVF and Alzheimer's Disease

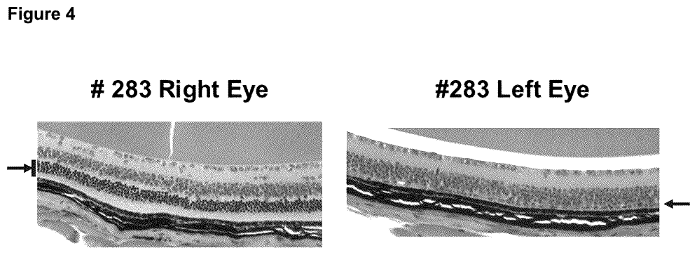

Rod derived cone viability factor (RdCVF), a trophic factor, was originally identified by screening mouse neural retina cDNA expression library for specific neuron-cone photoreceptor cell rescue. RdCVF has 2 isoforms owning to alternative splicing of nucleoredoxin-like (nxnl1) gene, the truncated form (RdCVFS) and full-length form (RdCVFL). Knockout of the nxnl1 gene caused mouse photoreceptor degeneration, suggesting that at genetic level this gene is essential for maintaining the survival of photoreceptors in mice. We have found intraocular administration of RdCVF1L mediated by adeno-associated viruses (AAV) significantly increased photoreceptor survival in a mouse model of inherited retinal degeneration (U.S. Pat. No. 9,265,813 and FIG. 4).

AAV2-RdCVF1L vectors were injected into the subretinal space of one eye of rd10 mice at postnatal day 3 while the fellow eye was left untouched to serve as a control. Mice were sacrificed at 10 weeks of age when retinal photoreceptor cells are completely degenerated without any treatment in this clinically relevant retinal degeneration model. Eyes were enucleated and further processed for histological analysis. Light microscopy showed approximately 5 layers of photoreceptor nuclei remained in the superior hemisphere of the vector-injected eyes (FIG. 4, Left Panel). In contrast, in untreated contralateral eyes, no or only a few photoreceptor nuclei were left in the retina (FIG. 4, Right Panel).

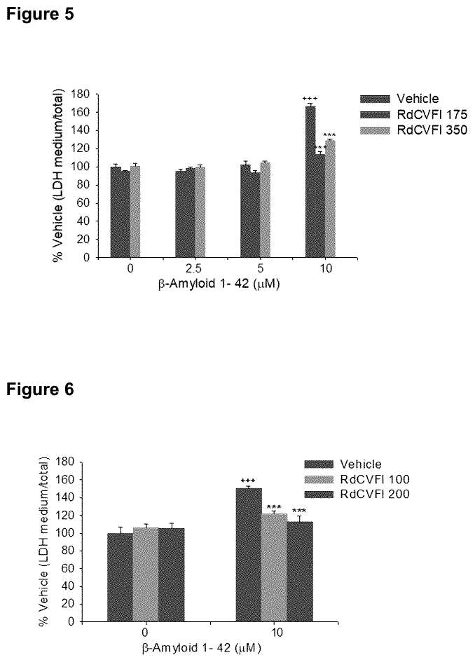

As mentioned above, we showed the neuroprotective effect of RdCVF1L in the retinal neurons. Since retina is an extension of the brain that resides in the back of the eye, we hypothesized the similar protection of RdCVF1L may apply to brain neurons. Hence, we selected 2 commercial-available human neuronal cell lines, human neuroblastoma and human neural progenitor cells to test this hypothesis. Surprisingly, we found RdCVF1L can protect these neuronal cells against 0-amyloid-induced toxicity (FIGS. 5 and 6).

SH-SY5Y, a human neuroblastoma cell line, was purchased from ATCC (Manassas, VA) and used for lactate dehydrogenase (LDH) cytotoxicity assay. LDH assay was performed using a CytoTox 96® Non-Radioactive Cytotoxicity Assay kit (Promega) to measure release of LDH from damaged cells.

Briefly, 1×106 of SH-SY5Y cells were seeded into 6-well plate overnight, the following day, regular DMEM medium was removed, and cells were treated with 500 μL of serum free DMEM medium containing 175, or 350 ng/mL RdCVF1L for 24 hrs. Then Aβ 1-42 (at 0, 2.5, 5, and 10 μM, Invitrogen) was added to each well for 24 hrs. 50 μL of the medium supernatant and cell lysate was used for LDH assay. Cytotoxicity was calculated relative to control cells. We found Aβ at 2.5 and 5 μM did not cause significant toxicity as compared to vehicle, but at 10 μM it induced significant increase in LDH release (P<0.001). Surprisingly, treatment with RdCVF1L at both concentrations significantly decreased the LDH release (P<0.001) induced by 10 μM Aβ as compared to vehicle treatment, indicating a neuroprotective effect by RdCVF1L (FIG. 5).

To further confirm RdCVF1L against Aβ neurotoxicity, we conducted a similar experiment using Human Neural Progenitor (NHNP) cells (Lonza, Walkersville, MD). NHNP cells were cultured to further differentiate into neuronal cells using the Neural Progenitor Differentiation Medium Bullet Kit (Lonza) containing the necessary supplements and media for optimal NHNP differentiation. Differentiated NHNP cells were cultured for 8 days, and incubated with RdCVF1L at 100 and 200 ng/mL or vehicle for 24 hours, then subjected to toxic treatment with Aβ at 10 μM for 2 days. LDH release was measured in media supernatant and cell lysate. Again, we surprisingly found RdCVF1L, at even lower concentrations, was able to significantly decrease Aβ-induced LDH release (P<0.001) as compared to vehicle, further corroborating its neuroprotective effect (FIG. 6).

The invention claimed is:

1. A nucleic acid comprising a nucleotide sequence encoding a full-length rod-derived cone viability factor (“RdCVF-long” or “RdCVFL”) protein and a nucleotide sequence encoding a human immunoglobulin kappa chain (IgK) signal sequence, wherein the human IgK signal sequence comprises an amino acid sequence with at least 95% sequence identity to SEQ ID NO: 7.

2. The nucleic acid of claim 1, wherein the RdCVFL protein is an RdCVF1L protein or an RDCVF2L protein.

3. (canceled)

4. (canceled)

5. The nucleic acid of claim 1, wherein the nucleotide sequence encoding the RdCVFL protein comprises a recoded nucleotide sequence.

6. (canceled)

7. The nucleic acid of claim 1, wherein the human IgK signal sequence is N-terminal to the RdCVFL protein.

8. (canceled)

9. The nucleic acid of claim 1, wherein the RdCVFL protein and the human IgK signal sequence comprise an amino acid sequence with at least 95% sequence identity to SEQ ID NO: 3 or SEQ ID NO: 15.

10-14. (canceled)

15. The nucleic acid of claim 1, wherein a promoter sequence is operatively linked to the nucleotide sequence encoding the human IgK signal sequence and human RdCVFL protein.

16-18. (canceled)

19. The nucleic acid of claim 1, wherein an intron sequence is operatively linked to the sequence encoding the RdCVFL protein.

20-22. (canceled)

23. A vector comprising the nucleic acid of claim 1.

24. (canceled)

25. The vector of claim 23, wherein the vector is a non-viral vector and the non-viral vector is selected from a lipid nanoparticle (LNP), highly branched poly(β-amino ester) (HPAE), single-chain cyclic polymer (SCKP), poly(amidoamine) (PAMAM) dendrimer, and polyethyleneimine (PEI).

26. (canceled)

27. The vector of claim 23, wherein the vector is a viral vector and the viral vector is selected from a retroviral vector, lentiviral vector, adenoviral vector, adeno-associated virus (AAV) vector, Herpes viral vector, hepatitis viral vector, SV40 vector, EBV vector and Newcastle disease virus vector.

28. The vector of claim 27, wherein the viral vector is an adeno-associated virus (AAV) vector.

29. The viral vector of claim 28, wherein the AAV vector is selected from AAV1, AAV2, AAV3, AAV4, AAV5, AAV6, AAV7, AAV8, AAV9 and AAVrh74.

30. The viral vector of claim 29, wherein the AAV vector is AAV2.

31. The viral vector of claim 29, wherein the AAV vector is AAV8.

32. (canceled)

33. An isolated cell comprising the nucleic acid of claim 1, wherein the cell is capable of secreting the RdCVFL protein.

34. A method for producing the RdCVFL protein comprising culturing the cell of claim 33 under conditions that allow for expression and secretion of the RdCVFL protein and isolating the RdCVFL protein from the cell culture.

35. (canceled)

36. A method of secreting an RdCVFL protein from a cell comprising administering to the cell the nucleic acid of claim 1 or a vector comprising the nucleic acid under conditions permitting expression and secretion of RdCVFL encoded by the nucleic acid or vector.

37-45. (canceled)

46. A pharmaceutical preparation comprising (i) a pharmaceutically acceptable carrier and (ii) the nucleic acid of claim 1, a vector comprising the nucleic acid, or (iii) a combination thereof.

47. A method of preserving ocular rod and cone cells in the eye of a mammal comprising administering to the eye of the mammal the nucleic acid of claim 1, a vector comprising the nucleic acid, a pharmaceutical composition comprising the nucleic acid or vector, or a combination thereof, in an amount effective to preserve the ocular rod and cone cells.

48. (canceled)

49. The method of claim 47, wherein
a) about 5×10⁸ to about 1×10¹¹ vector genome copy (GC) of an AAV vector is administered by subretinal injection;
b) wherein about 5×10⁸ to about 5×10¹² vector genome copy (GC) of an AAV vector is administered by intravitreal injection; or
c) wherein about 5×10⁸ to about 5×10¹⁴ vector genome copy (GC) of an AAV vector is administered by intrathecal injection.

50-52. (canceled)

53. The method of claim 47, wherein the mammal suffers from an ocular disease selected from the group consisting of a retinal dystrophy, Stargardt's disease, retinitis pigmentosa, dry age-related macular degeneration (dry AMD), geography atrophy (advanced stage of dry AMD), wet age-related macular degeneration (wet AMD), glaucoma/ocular hypertension, diabetic retinopathy, Bardet-Biedel syndrome, Bassen-Kornzweig syndrome, Best disease, choroidema, gyrate atrophy, congenital amaurosis, refsun syndrome, Usher syndrome, thyroid related eye disease, Grave's disease, a disease associated with retinal pigmented epithelial cells, anterior segment disease, lens disease/cataracts, an eye cup disorder, or uveitis.

54-56. (canceled)

57. A method of treating a disease comprising administering to a mammal the nucleic acid of claim 1, a vector comprising the nucleic acid, a pharmaceutical preparation comprising the nucleic acid or the vector, or a combination thereof, wherein the disease is a Central Nervous System (CNS) Disease.

58-63. (canceled)
